SEQUENCE LISTING (8 sequences)

SEQ ID NO: 1 - DNA, 20 bp, Artificial Sequence
  primer
gggctgcttttaactctggt

SEQ ID NO: 2 - DNA, 18 bp, Artificial Sequence
  primer
gcaggtttttctagacgg

SEQ ID NO: 3 - DNA, 23 bp, Artificial Sequence
  primer
tgaggagggctggaacaagtacc

SEQ ID NO: 4 - DNA, 23 bp, Artificial Sequence
  primer
ggaggtggtaattgcagggaaca

SEQ ID NO: 5 - DNA, 20 bp, Artificial Sequence
  primer
ggacacaatggattgcaagg

SEQ ID NO: 6 - DNA, 20 bp, Artificial Sequence
  primer
taaccactgctccactctgg

SEQ ID NO: 7 - DNA, 20 bp, Artificial Sequence
  primer
atctgaagaaggagagcgag

SEQ ID NO: 8 - DNA, 20 bp, Artificial Sequence
  primer
tcagaagtctccagagcttg

(19) United States
(12) Patent Application Publication
(10) Pub. No.: US 2011/0014267 A1
(43) Pub. Date: Jan. 20, 2011

(54) BIOSYNTHETIC CARTILAGINOUS MATRIX AND METHODS FOR THEIR PRODUCTION

(76) Inventors: Hanne Everland, Bagsvaerd (DK); Peter Samuelsen, Rungsted Kyst (DK); Jakob Vange, Helsingoer (DK); Christian Clausen, Fredensborg (DK)

Correspondence Address: JACOBSON HOLMAN PLLC, 400 SEVENTH STREET N.W., SUITE 600, WASHINGTON, DC, 20004, US

(21) Appl. No.: 12/735,978

(22) PCT Filed: Mar. 2, 2009
(86) PCT No.: PCT/EP2009/052432

§ 371 Date: Aug. 27, 2010

(30) Foreign Application Priority Data
Feb. 29, 2008 (DK) PA 2008 00308

(51) Int. Cl. A61K 9/00; C12N 5/077; A61K 35/12; A61P 43/00

(52) U.S. Cl. 424/426; 435/395; 435/396; 424/422; 424/93.7

(57) ABSTRACT

An isolated, acellular biosynthetic cartilaginous matrix substantially devoid of synthetic biodegradable scaffold structure is provided. Through a method with the steps of a) contacting in vitro a population of chondrogenic cells with a synthetic biodegradable scaffold; b) culturing in vitro for a period of time said chondrogenic cells within said synthetic biodegradable scaffold so that the chondrogenic cells produce a biosynthetic cartilaginous matrix; c) substantially removing any antigen derived from said chondrogenic cells a matrix suitable of implantation into a living individual mammal, such as a human being is obtained.

### FIELD OF THE INVENTION

The present invention relates to a biosynthetic cartilage, methods for the in vitro preparation of such cartilage suitable for in situ cartilage repair, as well as methods of treatment.

### BACKGROUND OF THE INVENTION

Tissue engineering methods using cell transplantation are known, and for example, may involve for instance open joint surgery (e.g. open knee surgery) and, in case of joint surgery, extensive periods of relative disability for the patient to recuperate in order to ensure that optimal results are achieved. Such procedures are costly, and require extensive medical procedures such as rehabilitation and physical therapy.

Methods using scaffold technologies of various forms, where the scaffold (with, or without cells grown in the scaffold) is inserted into the defect, have suffered from difficulties in performing the cell implantation procedure solely guided by arthroscopy.

Arthroscopic Autologous Cell Implantation (called AACI or ACI using minor surgical interventions) is a surgical procedure for treating cartilage or bone defects, whereby a scaffold is inserted into the defect concomitantly with applying cell suspension or cell mixture with precursor fixatives, into said defect using a needle as for instance a “blunt” needle or a catheter. This implantation procedure is visualized and guided by an arthroscope.

WO 2004/110512 discloses an endoscopic method, useful for treating cartilage or bone defects in mammals, involving identifying the position of defect and applying chondrocytes, chondroblasts, osteocytes and osteoblasts cells into cartilage or bone defect. The cells are applied with a solidafiable support material, such as soluble thrombin and fibrinogen or collagen mixtures. It is envisaged that, for surgery in a convex or concave joint, that a porous membrane may be placed at the site of defect, but removed once the fibrin/cell mix are coagulated in place. The method disclosed in WO 2004/110512 allows tissues to be repaired arthroscopically, i.e. without the need of open joint surgery (e.g. open knee surgery).

Scaffolds are porous structures into which cells may be incorporated. They are usually made up of biocompatible, bio-degradable materials and are added to tissue to guide the organization, growth and differentiation of cells in the process of forming functional tissue. The materials used can be either of natural or synthetic origin.

WO 2007/028169 relates to a method for tissue engineering by cell implantation that involves the use of a scaffold in situ at the site of a defect, where the therapeutic cells are fixed in place into the scaffold only once the scaffold is inserted at the site of the tissue defect.

WO 2007/101443 provides preferred scaffold materials for use in the methods and kit of parts of the present invention.

The present invention provides new and improved biosynthetic cartilaginous matrix as well as methods for in vitro preparation of such chondrogenic matrix in a solid scaffold system. Further provided are methods for improved in situ cartilage repair, wherein such in vitro prepared chondrogenic matrix are incorporated into the site of a cartilage defect.

### BRIEF DESCRIPTION OF FIGURES

FIG. 1: Histology staining according to example 1. The figure show representative microphotographs of hAC-loaded MPEG-PLGA 6 weeks and 12 weeks old scaffolds after staining. Sections are staining with TB and SO; TB=Toluidine Blue; SO=Safranin O.

FIG. 2: IHC analysis according to example 1. The IHC analysis confirmed the findings with TB and SO, and demonstrated that the essential chondrogenic markers, aggrecan and collagen type II, for normal articular cartilage tissue are present within the scaffold structure after culture.

FIG. 3: RT-PCR analysis according to example 1. In the RT-PCR analysis an upregulation of the collagen type II and aggrecan was observed depending on time in culture. Furthermore the transcription factor necessary for driving and maintaining the hACs in the chondrocyte lineage was present and furthermore upregulated.

FIG. 4: Migration analysis (Unstained and TB-stained) according to example 1. The figure illustrates the migration out of the central part of the MPEG-PLGA scaffold system, observed after 5 days.

FIG. 5: The figure illustrates the results according to example 2.

FIG. 6: The figure illustrates the results according to example 4. Molecular weight of degraded samples

FIG. 7: The figure illustrates the results according to example 4. Normalized area of degraded samples

### SUMMARY OF THE INVENTION

It has surprisingly been found by the inventors of the present invention that a complete biosynthetic cartilaginous matrix suitable of implantation into a living individual mammal, such as a human may be prepared entirely by in vitro methods. It is to be understood that the present methods for the preparation of a cartilaginous matrix is an in vitro method, i.e. a method, which is independent on any in vivo conditions. Thus, the cartilaginous matrix is made into a complete biosynthetic cartilaginous matrix outside the living individual mammal, such as the human body before being implanted into the living individual for restoration of a cartilage defect.

In a broad aspect the present invention provides methods for the preparation of acellular and/or antigen-free biosynthetic tissues, such as cartilaginous matrix.

In another broad aspect the present invention provides methods for the preparation of biosynthetic tissues, such as cartilaginous matrix substantially devoid of synthetic biodegradable scaffold structures. Accordingly, in some embodiments of the invention, this biosynthetic cartilaginous matrix primarily contains biologically derived material, i.e. material produced by a mammal cells, such as a chondrocyte.

In a first aspect the invention provides a method for the preparation of a biosynthetic cartilaginous matrix suitable of implantation into a living individual mammal, such as a human being, the method comprising the sequential steps of:
a) contacting in vitro a population of chondrogenic cells with a synthetic biodegradable scaffold;
b) culturing in vitro for a period of time the chondrogenic cells within the synthetic biodegradable scaffold so that the chondrogenic cells produce a biosynthetic cartilaginous matrix.

In a second aspect the invention provides a method for the preparation of a biosynthetic cartilaginous matrix suitable of implantation into a living individual mammal, such as a human being, the method comprising the sequential steps of:
a) contacting in vitro a population of chondrogenic cells with a synthetic biodegradable scaffold;
b) culturing in vitro for a period of time the chondrogenic cells within the synthetic biodegradable scaffold so that the chondrogenic cells produce a biosynthetic cartilaginous matrix; wherein during any one of steps a)-b) and/or in a subsequent step the biodegradable scaffold is completely or partially degraded in vitro.

In a third aspect the invention provides a method for the preparation of a biosynthetic cartilaginous matrix suitable of implantation into a living individual mammal, such as a human being, the method comprising the sequential steps of:
a) contacting in vitro a population of chondrogenic cells with a synthetic biodegradable scaffold;
b) culturing in vitro for a period of time the chondrogenic cells within the synthetic biodegradable scaffold so that the chondrogenic cells produce a biosynthetic cartilaginous matrix; and
c) substantially removing any antigen derived from the chondrogenic cells.

In a further aspect the invention provides a method for the preparation of a biosynthetic cartilaginous matrix suitable of implantation into a living individual mammal, such as a human being, said method comprising the sequential steps of:
a) contacting in vitro a population of chondrogenic cells with a synthetic biodegradable scaffold;
b) culturing in vitro for a period of time said chondrogenic cells within said synthetic biodegradable scaffold so that the chondrogenic cells produce a biosynthetic cartilaginous matrix;
c) substantially removing any antigen derived from said chondrogenic cells;
   wherein during any one of steps a)-c) and/or in a subsequent step the biodegradable scaffold is completely or partially degraded in vitro.

In a further aspect the invention provides for a biosynthetic cartilaginous matrix prepared by a method comprising the sequential steps of:
a) contacting in vitro a population of chondrogenic cells with a synthetic biodegradable scaffold;
b) culturing in vitro for a period of time the chondrogenic cells within the synthetic biodegradable scaffold so that the chondrogenic cells produce a biosynthetic cartilaginous matrix;
c) substantially removing the chondrogenic cells;
   wherein during any one of steps a)-c) and/or in a subsequent step the biodegradable scaffold is completely or partially degraded in vitro.

In a further aspect the present invention provides an isolated acellular biosynthetic cartilaginous matrix substantially devoid of synthetic biodegradable scaffold structure. In one embodiment, the isolated acellular biosynthetic cartilaginous matrix has a morphological structure substantially comparable with the morphological structure of the synthetic biodegradable scaffold used according to the invention. Accordingly, the acellular biosynthetic cartilaginous matrix may have size, shape or other morphological features according to the synthetic biodegradable scaffold used according to the invention.

The term “isolated”, as used above, refers to the biosynthetic cartilaginous matrix being isolated from other components, such as isolated from e.g. tissue of a mammal having an implant. Accordingly a potential acellular biosynthetic cartilaginous matrix made in situ in a live individual mammal is preferably not within the scope of the present invention.

In a further aspect, the present invention provides a method for the treatment or for alleviating the symptoms of a cartilage defects in a living individual mammal, such as a human being, the method comprising the step of applying an acellular biosynthetic cartilaginous matrix substantially devoid of synthetic biodegradable scaffold structure to the site of the defect.

In a further aspect, the present invention provides an acellular biosynthetic cartilaginous matrix, such as a biosynthetic cartilaginous matrix suitable for use as an implant, substantially devoid of synthetic biodegradable scaffold structure; for use as a medicament.

In a further aspect, the present invention provides an acellular biosynthetic cartilaginous matrix, such as a biosynthetic cartilaginous matrix suitable for use as an implant, substantially devoid of synthetic biodegradable scaffold structure; for the preparation of a medicament.

In a further aspect, the present invention provides kit of parts, for the treatment or for alleviating the symptoms of a cartilage defects in a living individual mammal, the kit comprising an acellular biosynthetic cartilaginous matrix and instructions for use of the biosynthetic cartilaginous matrix.

### DETAILED DESCRIPTION OF THE INVENTION

As described above an important aspect of the present invention is a method for the preparation of a biosynthetic cartilaginous matrix suitable of implantation into a living individual mammal, such as a human being, the method comprising the sequential steps of:
a) contacting in vitro a population of chondrogenic cells with a synthetic biodegradable scaffold;
b) culturing in vitro for a period of time the chondrogenic cells within the synthetic biodegradable scaffold so that the chondrogenic cells produce a biosynthetic cartilaginous matrix;
c) substantially removing any antigen derived from the chondrogenic cells;
   wherein during any one of steps a)-c) and/or in a subsequent step the biodegradable scaffold is completely or partially degraded in vitro.

In previous disclosures made by K. Osther and others (e.g. WO9808469; WO02083878 WO03028545 and U.S. Pat. Nos. 5,759,190; 5,989,269; 6,120,514; 6,283,980; 6,379,367; 6,592,598; 6,592,599; 6,599,300; 6,599,301), the cells are applied in the scaffold and cultured into the scaffold for some time prior to placing both the cells and the scaffold containing the cells in the target (e.g. cartilage defect).

However, the present invention, wherein a biosynthetic cartilaginous matrix is made in vitro results in improved, more convenient, and less expensive procedures.

Important aspects of the present invention are the removal of antigens derived from the chondrogenic cells from the biosynthetic cartilaginous matrix. Accordingly, the inventors of the present invention have found methods wherein a biosynthetic cartilaginous matrix may be produced in high-scale amounts suitable for implantation not only into individuals, where the cells are derived from, but also into other individual mammals, without the risk of an immunological response to foreign cell antigens.

Accordingly cells from any human being or from any non-human mammal species, such as a pig may be used to prepare the biosynthetic cartilaginous matrix suitable for implantation into any other human being or any other mammal species.

When the term “about” is used herein in conjunction with a specific value or range of values, the term is used to refer to both about the range of values, as well as the actual specific values mentioned.

The term “substantially removing any antigen” as used herein refers to the complete or partial removal of chondrogenic cell antigens to a level, wherein no significant or serious immunological response by the living individual mammal receiving the implant, irrespective of the source of the chondrogenic cells.

In some embodiments according to the invention, the removal of antigens is performed by substantially removing the population of chondrogenic cells, from said biosynthetic cartilaginous matrix.

Whilst the removal of the cells is performed to reduce or eliminate the risk of implant rejection and to ensure immunocompatibility of cartilage implants, in one embodiment, the substantial removal of the population of chondrogenic cells from said biosynthetic matrix may be determined by a quantitative PCR determination of the DNA or RNA molecules of the cells so that the removal results in a decrease in the signal from quantitative PCR by at least 30%, such as at least 40%, at least 50%, such as at least 60%, such as at least 70%, such as at least 70%, such as at least 80%, such as at least 90%, such as at least 95%, such as at least 98%, such as at least 99% or about 100%. Alternatively, the substantial removal of the population of chondrogenic cells from said biosynthetic matrix may be determined by histochemical staining of cells present in the matrix before and after removal of the cells, so that the removal results in a decrease in the number of cells by at least 30%, such as at least 40%, such as at least 50%, such as at least 60%, such as at least 70%, such as at least 70%, such as at least 80%, such as at least 90%, such as at least 95%, such as at least 98%, such as at least 99% or about 100% (i.e. essentially acellular).

In one embodiment, the substantial removal of the population of chondrogenic cells from said biosynthetic matrix may be determined by a PCR determination using the method according to example 9.

The term “removing the population of chondrogenic cells” as used herein refers to the removal of whole chondrogenic cells as well as, preferably, potentially antigenic membrane or intracellular proteins derived from the chondrogenic cells. Included within this definition are cell membrane proteins or intracellular proteins that may be present within the biosynthetic cartilaginous matrix after e.g. chondrocyte cell lysis during chondrocyte cell removal.

This may be accomplished by incubation of the biosynthetic cartilaginous matrix in a suitable solution providing the matrix with an enzymatic treatment, nuclease treatment, hypertonic or hypotonic treatment, ionic or non-ionic detergent treatment, such as a solution comprising TRIS or Triton X-100, as described in example 6.

Other important aspects of the present invention are the removal of synthetic biodegradable scaffold material from the biosynthetic cartilaginous matrix. Accordingly the present invention provides a biosynthetic cartilaginous matrix, wherein the matrix polymers preferably consist mainly or only of biologically derived materials.

It is to be understood that there may be remnants or degradation products of the synthetic biodegradable scaffold. However, preferable these will not be a significant portion of the biosynthetic cartilaginous matrix.

The inventors of the present invention expect that at complete biosynthetic cartilaginous matrix substantially without synthetic biodegradable scaffold material and accordingly substantially consisting entirely of biologically derived cartilaginous materials may treat or alleviate the symptoms of a cartilage defects faster than for implants known in the art.

The term “contacting in vitro”, as used herein, refers to the step of the method according to the invention, wherein chondrogenic cells are applied onto, together with or within the scaffold under in vitro conditions, i.e. under conditions of a controlled environment outside of a living mammal.

The term “culturing in vitro”, as used herein, refers to the step of the method according to the invention, wherein chondrogenic cells are maintained under in vitro conditions, i.e. under conditions of a controlled environment outside of a living mammal. Alternatively the skilled person may use the phrases that the “cells are grown”, or “cells are proliferated” in vitro, which is also within the meaning of “culturing”.

In particular aspects, the chondrogenic cells mixed with culture medium are placed on the surface of or at least in conjunction with the scaffold, usually in a culture dish or flask. The chondrogenic cells may be placed together with a component which facilitates the cell adhesion and/or in-growth are absorbed through scaffold.

The methods described may be applied using any chondrogenic cells for the preparation of a biosynthetic cartilage matrix suitable for the treatment of any cartilage defects.

The term “biosynthetic cartilaginous matrix” as used herein is intended to mean the matrix comprising connective tissue and/or extracellular matrix components produced by chondrogenic cells in vitro, which matrix is suitable of implantation into a living individual mammal.

It is to be understood that once the chondrogenic cells have been applied to the synthetic biodegradable scaffold, the cells are allowed to migrate and/or grow through the scaffold to generate a new biosynthetic cartilaginous matrix. In one embodiment a component which facilitates cell adhesion and/or in-growth is concomitantly applied to the scaffold.

In an important embodiment of the invention, the method for the preparation of a biosynthetic cartilaginous matrix comprises a step of substantially removing the population of chondrogenic cells, or remnants of cells, from the biosynthetic cartilaginous matrix.

In one embodiment the biosynthetic cartilaginous matrix potentially comprising synthetic biodegradable scaffold will after this step to be essentially cell free.

The term “essentially cell free”, refers to a biosynthetic cartilaginous matrix that does not comprise the living mammalian chondrogenic cells prior to use in the method according to the invention. In one embodiment, the term “essential cell free” is equivalent to “cell free”, and means that the scaffold is sterile, and comprises no living micro-organism or mammalian cells which could survive and/or replicate once introduced into the patient, preferably no living cells whatsoever.

In some important aspects of the invention, the synthetic biodegradable scaffold is completely or partially degraded in vitro during the step, wherein the chondrogenic cells are cultured within the synthetic biodegradable scaffold or in a subsequent step.

It is to be understood that after this complete or partial degradation of the synthetic biodegradable scaffold, only or at least mainly the biosynthetic cartilaginous matrix and potentially also chondrogenic cells will be left.

The term “completely or partially degraded in vitro” refers to a step wherein the synthetic biodegradable scaffold is degraded by the action of some intrinsic or extrinsic component of the in vitro system. This action may be endogenous enzymatic activity of the chondrogenic cells or alternatively by the activity of compounds added during the cell culturing, such as in the medium, or in a subsequent step. Alternatively, it may be auto-degradation due to the intrinsic action of free radicals of the synthetic biodegradable scaffold material.

In some embodiments the synthetic biodegradable scaffold is degraded to a level wherein the ratio as measured by weight percent between the biosynthetic cartilaginous matrix and the synthetic biodegradable scaffold is within the range of 1000:1 to 10:1, such as higher than 100:1.

In some embodiments the synthetic biodegradable scaffold is completely or partially degraded by free radical degradation, i.e. degraded by the action of radicals, such as radicals in the scaffold material itself.

It is to be understood that the scaffold material, with an inherent rate of autodegradation due to e.g. radical degradation, may be selected to fit the time necessary for the chondrogenic cells to produce the biosynthetic cartilaginous matrix.

In some embodiments the synthetic biodegradable scaffold is completely or partially degraded by application of irradiation, such as high dose irradiation.

In some embodiments the synthetic biodegradable scaffold is completely or partially degraded by cellular degradation, i.e. degraded by the action of cell enzymes.

In some embodiments the synthetic biodegradable scaffold is completely or partially degraded by hydrolysis, i.e. when contact with water.

It is to be understood that the scaffold material sensitive to cellular degradation may be selected to fit the time necessary for the chondrogenic cells to produce the biosynthetic cartilaginous matrix.

It is to be understood that the time needed for degradation of the scaffold material may be significantly reduced by the application of irradiation, enzymes, acids or alkaline solutions.

In some embodiments the synthetic biodegradable scaffold is sterilised through the application of irradiation, such as beta radiation, or plasma sterilisation; prior to in vitro application of chondrogenic cells to the scaffold.

In some embodiments according to the invention, step a) and/or step b) as described above further comprises administering a component which facilitates the cell adhesion and/or in-growth for generation of biosynthetic cartilaginous matrix within the synthetic biodegradable scaffold, such as an extracellular matrix component of any suitable tissue, such as extracellular matrix components from bladder, intestine, skin.

Accordingly, in some embodiments according to the invention, step a) and/or step b) as described above further comprises administering a component which facilitates the cell adhesion and/or in-growth for generation of biosynthetic cartilaginous matrix within the synthetic biodegradable scaffold, such as a component selected from the group consisting of: chondroitin sulfate, hyaluronan, hyaluronic acid (HA), heparin sulfate, heparan sulfate, dermatan sulfate, growth factors, fibrin, fibronectin, elastin, collagen, such as collagen type I and/or type II, gelatin, and aggrecan, or any other suitable extracellular matrix component.

In one particular embodiment, hyaluronic acid is incorporated into said synthetic biodegradable scaffold. In one embodiment, the hyaluronic acid is present in said synthetic biodegradable scaffold at a proportion of between about 0.1 and about 15 wt %.

In a further specific embodiment, dermatan sulphate is incorporated into said synthetic biodegradable scaffold. In one embodiment the dermatan sulphate is present in said synthetic biodegradable scaffold at a proportion of between about 0.1 and about 15 wt %.

In some embodiments according to the invention, step a) and/or step b) as described above further comprises administering a suspension of extracellular matrix components produced by a chondrogenic cells. This may be usually be suspension of extracellular matrix components produced by a chondrogenic cells in vitro.

In other embodiments according to the invention, step a) and/or step b) as described above further comprises administering a suspension of extracellular matrix components produced by a chondrogenic cells together with these chondrogenic cells.

In still other embodiments according to the invention, step a) and/or step b) as described above further comprises administering a tissue explant from the recipient of the biosynthetic cartilaginous matrix suitable of implantation, the explant comprising extracellular matrix components and chondrogenic cells derived from this recipient.

The inventors of the present invention have found that a suspension of extracellular matrix components produced by a chondrogenic cells added to the synthetic biodegradable scaffold may facilitate and increase the speed of formation and size of formed biosynthetic cartilaginous matrix.

In some embodiments according to the invention, step a) and/or step b) as described above further comprises administering a further compound to the synthetic biodegradable scaffold, wherein said further compound is selected from the group consisting of: growth factors, such as Insulin-like growth factor 1 (IGF-1), or Transforming growth factors (TGFs), such as TGF-alpha or TGF-beta, or FGFs, such as FGF-1 or FGF-2.

The terms “chondrogenic cells” or “chondrogenic cell”, refers to any cell that are obtained from or derived from a mammalian tissue, which may be maintained or cultured in vitro and which are or may be developed into a chondrocyte.

In one embodiment, the cells are obtained from or derived from the living individual mammal, where implantation is performed, i.e. are autologous.

The cells may also be homologous, i.e. compatible with the tissue to which they are applied, or may be derived from multipotent or even pluripotent stem cells, for instance in the form of allogenic cells. In one embodiment, the cells are non-autologous. In one embodiment, the cells are non-homologous. In one embodiment the cells may be allogenic, from another similar individual, or xenogenic, i.e. derived from an organism other than the organism being treated. The allogenic cells could be differentiated cells, progenitor cells, or cells whether originated from multipotent (e.g. embryonic or combination of embryonic and adult specialist cell or cells, pluripotent stemcells (derived from umbilical cord blood, adult stemcells, etc.), engineered cells either by exchange, insertion or addition of genes from other cells or gene constructs, the use of transfer of the nucleus of differentiated cells into embryonic stemcells or multipotent stem cells, e.g. stem cells derived from umbilical blood cells.

It is to be understood that one important aspect of the present invention is the substantial removal of any antigen derived from the cells used to produce the biosynthetic cartilaginous matrix. Thus, chondrogenic cells, which are not normally compatible with the tissue to which the biosynthetic cartilaginous matrix may be applied, may be used, in particular, where the use of such cells have other advantages, such as availability, growth rate or ability to produce the biosynthetic cartilaginous matrix.

In one embodiment, the method of the invention also encompasses the use of stem cells, and cells derived from stem cells, the cells may be, preferably obtained from the same species as the individual mammal being treated, such as human stem cells, or cells derived there from.

The chondrogenic cells may be prepared as described in WO 02/061052, which is hereby incorporated by reference.

The chondrogenic cells are typically mammalian chondrogenic cells, which in some embodiments are obtained or derived from said individual mammal being treated according to the invention. Such methods of obtaining and culturing cells from the individual mammal are disclosed in WO 02/061052.

The mammalian chondrogenic cells may be supplied in the form of a cell suspension or tissue explants. Tissue explants may be directly taken from other parts of the individual mammal, and may therefore be in the form of tissue grafts such as a knee meniscal graft.

The mammalian chondrogenic cells may be any chondrogenic cell suitable to produce biosynthetic cartilaginous matrix. Suitable chondrogenic cells may include a cultured chondrocyte, such as a cultured knee meniscal chondrocyte, chondrocyte-derived cell line such as CHON-001, CHON-002 (ATCC® Number: CRL-2846™, CRL-2847™), or TC28 cells, or chondrogenic cells as disclosed in US patent applications US20050129673, US20060148077, US20030064511, US20020094754, U.S. Pat. No. 6,841,151, U.S. Pat. No. 6,558,664, and in U.S. Pat. No. 6,340,592.

Human articular chondrocytes are particularly preferred.

It is envisaged that stem cells, or any other suitable precursor cells which are capable of becoming or producing chondrocytes may also be used.

Typically, the cells used in the second component are present in a sufficient amount of cells to result in regeneration or repair of the target tissue or defect, such as of about 0.1×10⁴ to about 10×10⁶ cells/ml, or 0.1×10⁶ cells/ml to about 10×10⁶ cells/ml.

Prior to use, the chondrogenic cells are typically placed in a suitable suspension with a culture media, which may optionally comprise growth hormones, growth-factors, adhesion-promoting agents, and/or physiologically acceptable ions, such as calcium and/or magnesium ions (see WO 2004/110512). It is highly preferably that the cell suspension does not comprise significant levels of blood serum, i.e. are essentially serum free, such as free of autologous or homologous blood serum, particularly if the serum contains components which may interfere with the formation of the fixative in situ at the defect site.

In some embodiments the population of chondrogenic cells used in the methods according to the invention is selected from the list consisting of chondrocytes, such as human articular chondrocytes, stem cells or equivalent cells capable of transformation into a chondrocyte, such as mesenchymal stem cells or embryonic stem cells.

In some embodiments the chondrogenic cells used according to the invention are non-autologous and/or non-homologous relative to the living individual mammal, wherein the cartilaginous matrix is implantated.

One important problem solved by the present invention is to provide implants of cartilaginous matrix, which is not sensitive to the source of the cells. Accordingly antigenicity issues associated with origin of these cells have been solved by providing a biosynthetic cartilaginous matrix suitable of implantation, which is essentially free of antigens derived from the host cells.

In some embodiments the chondrogenic cells used according to the invention are in the form of a cell suspension, cell associated matrix, or tissue explant.

In some embodiments the chondrogenic cells are introduced under step a), of the method according to the invention, in an amount of about 0.1×10⁴ cells to about 10×10⁶ cells per 0.1 cm³ of synthetic biodegradable scaffold.

In some embodiments the chondrogenic cells are cultured under step (b), of the method according to the invention, for a period of at least 1 week, such as at least 2 weeks, such as at least 3 weeks, such as at least 6 weeks, such as at least 12 weeks.

The “living individual mammal” is any living individual mammal suitable for implantation, and is preferably a human being, typically a patient. However the methods of the invention may also be applicable to other mammals, such as a dog, a horse or a goat.

The methods for implantation of the biosynthetic cartilaginous matrix according to the invention may be performed as, or during a method of surgery, such as a method of endoscopic, arthroscopic, or minimal invasive surgery, or conventional or open surgery.

In one embodiment, the implantation is performed during reconstruction surgery or cosmetic surgery.

The term “defect” as used herein refers to any detrimental or injured condition of a tissue, which is associated with existing, or future, loss of, or hindered function, disability, discomfort or pain. The defect is preferably associated with a loss of normal tissue, such as a pronounced loss of normal tissue. It is envisaged that the methods of the invention may be used prophylactically, i.e. to prevent the occurrence of defects, or for preventing the deterioration of an existing defect. The defect may, for example be a cavity in the tissue, a tear or wound in the tissue, loss of tissue density, development of aberrant cell types, or caused by the surgical removal of non-healthy or injured tissue etc. In a preferred embodiment, the defect could either an injured articular cartilage, an articular cartilage defect down to and/or involving the bone (osteoarthritis), a combination of cartilage and bone defect, a defect in bone which is surrounded by normal cartilage or bone, or a defect in a bone structure itself or be a bone structure that needs re-inforcement by addition of bone cells with scaffold as in the SCAS system. In a most preferred embodiment, the defect is in cartilage, such as articular cartilage defect.

The term “tissue” as used herein refers to a solid living tissue which is part of a living mammalian individual, such as a human being. The tissue may be a hard tissue (e.g. bone, joints and cartilage). The tissue may be selected from the group consisting of: cartilage, such as articular cartilage, bone, skin, ligament, tendon, and other mesenchymal tissues.

It is important to understand that the biosynthetic cartilaginous matrix may not only be used to cartilage defects as such, but may be used in any surgical situation, where biosynthetic cartilaginous matrix is required. This may be any cosmetic or reconstructural surgical situation.

One important aspect of the invention relates to a method for the treatment or for alleviating the symptoms of a cartilage defects in a living individual mammal, such as a human being, said method comprising the step of applying a biosynthetic cartilaginous matrix according to the invention to the site of a defect or place requiring implantation.

As described above another important aspect of the present invention relates to an acellular biosynthetic cartilaginous matrix substantially devoid of synthetic biodegradable scaffold structure; for use as a medicament

In one embodiment this biosynthetic cartilaginous matrix according to the inventions is for use in the treatment or for alleviating the symptoms of a cartilage defects in a living individual mammal, such as a human being.

In some specific embodiments cells derived from the living individual mammal to have implantation are applied to the biosynthetic cartilaginous matrix prior to and/or concomitantly with and/or subsequent to the application of the biosynthetic cartilaginous matrix to the site of defect. It is expected by the inventors of the invention that this may facilitate the uptake and tolerance of the biosynthetic cartilaginous matrix, and thereby increase speed of recovery for the mammal being treated with the implant, such as a human patient.

In some embodiments one or more microfractures is purposely induced under clinical conditions at the site of implantation prior to application of the biosynthetic cartilaginous matrix. It is expected that host cells from the mammal being treated will migrate from the microfractures to assist the implant in attachment to this implantation site.

In some embodiments the biosynthetic cartilaginous matrix, such as in form of a disc, may be implanted in conjunction with or with access to cells, such as cells of the mammal host receiving the implant, e.g. by the induction of a microfracture. Alternatively, the biosynthetic cartilaginous matrix may be implanted in conjunction with or with access to allogenic or autologous cells relative to the mammal host receiving the implant.

In some embodiments the cartilage defect being treated is due to trauma, osteonecrosis, or osteochondritis, and located in a joint, such as in the knee joint, or located in the ankle, shoulder, elbow, hip or spinal cord.

In one important embodiment, the biosynthetic cartilaginous matrix is immuno-compatible with the living individual mammal to be treated.

In some embodiments the treatments according to the invention is performed as part of surgery, such as of endoscopic, atheroscopic, or minimal invasive surgery, and conventional or major open surgery.

In some embodiments the treatments according to the invention is performed as part of reconstruction surgery or cosmetic surgery.

As described elsewhere the present invention also provides kit of parts, for the treatment or for alleviating the symptoms of a cartilage defects in a living individual mammal, the kit comprising an acellular biosynthetic cartilaginous matrix and instructions for use of the biosynthetic cartilaginous matrix.

In one embodiment this kit comprises an integrated supply device, comprising the following functionally linked devices: (i) at least one container which contains said biosynthetic cartilaginous matrix according to the present invention, and (ii) a delivery device, wherein said delivery device is suitable for direct application of said biosynthetic cartilaginous matrix to the site of defect in a living mammalian tissue.

In some aspects of the invention, the kit further comprises a fixative. This fixative can be a suture, a stabler and/or tissue glue such as fibrin glue.

In one particular embodiment this delivery device is in the form of a medical device selected from the group consisting of: a syringe, a catheter, a needle, and a tube, a spraying device and a pressure gun. In another embodiment, the delivery device is an arthroscopic delivery device.

In one embodiment, chondrogenic cells are locked into the scaffold due to the cell culture medium and a gelating (fixative) material being added simultaneously or essentially concurrently, to the cell-free scaffold (or membrane). The cell-containing culture medium applied to the cell-free scaffold (or membrane) may therefore be dispersed simultaneously or essentially concurrently, with the gelating material which is also applied as a fluid to the scaffold.

The terms “fixative material” or “gelating material” as used herein thus refers to material suitable to fix or crosslink cells in the scaffold structure.

A preferred fixative material is fibrin.

In a preferred embodiment, the fixative material is in the form of a hydrogel, i.e. a gelating material capable of binding water, for example fibrin formed by the combination of the fixative precursor fibrinogen and the conversion agent thrombin.

The term “fixative precursor” as used herein refers to a compound or material that may be converted into a fixative material, usually by the action of another compound termed herein the “conversion agent”.

In one embodiment, the conversion agent may be a cross-linking agent and/or a polymerization agent and/or gelating agent.

In a preferred embodiment the conversion of the fixative precursor to the fixative occurs via the application of a conversion agent. The addition of the conversion agent to the fixative precursor, preferably occurs immediately prior to, simultaneous to, or immediately after the addition of the chondrogenic cells to the scaffold—i.e. the effect of the conversion agent in converting the fixative precursor to a fixative, such as a gel/hydrogel or solid, occurs only once the cells are in place, and typically when the cells have been distributed through the scaffold. The order of application of fixative precursor, conversion agent and chondrogenic cells are not essential. It is only important that they are kept separate prior to the method of the invention, therefore allowing concomitant, or essentially simultaneous, application during the method of the invention.

In one embodiment, the conversion agent is enzyme suitable of converting a substrate into a gel, such as a fibrin gel.

In one embodiment, the conversion agent is lyophilized with said biologically acceptable scaffold.

The scaffold preferably being hydrophilic by itself or by application of a hydrophilic solution then facilitates a “suction” of the combined cell fluid and fixative precursor and conversion agent into the scaffold, whereby the chondrogenic cells are locked and adhered to the scaffold.

In some aspects of the invention, the chondrogenic cells are applied and/or grown in the presence of a fixative precursor and conversion agent (e.g. fibrinogen mixed with a conversion agent, such as thrombin).

The conversion agent thrombin may be incorporated into the scaffold and the hydrogel will be formed when adding the fibrinogen/cell suspension to the scaffold.

In one embodiment, the scaffold is prepared in such a manner that it, prior to use, is “impregnated” with a fixative precursor and/or conversion agent, which is capable of retaining its activity (e.g. the thrombin analogues developed by HumaGene Inc., Chicago, Ill.). The scaffold is typically cut or shaped into the size of the defect, the chondrogenic cells, mixed with the fixative precursor and/or conversion agent (e.g. fibrinogen), are placed on the scaffold, which mixture when added to the scaffold, impregnated with another fixative precursor and/or conversion agent (e.g. thrombin analogue), will render the fixative precursor already in the scaffold active, thereby enabling it to react with the fixative precursor and/or conversion agent added together with the chondrogenic cells, resulting in gelation, clotting and adhesion.

The fixative precursor used in some embodiments of the invention may be any form of biocompatible glue or adhesive, including gelation agents, which are capable of being absorbed by the porous scaffold and, when converted into the fixative capable of anchoring both the cartilaginous matrix to the scaffold and the cells to the scaffold.

WO 2004/110512, which is hereby incorporated by reference, provides several fixative precursors and specific examples of suitable combinations of fixative precursors and conversion agents. Suitably, the ratio of fixative precursor to conversion agent may be used to control both the rate at which the fixation occurs, and the level of support provided by the fixative.

Suitable fixative precursors may be a polysaccharide such as agarose or alginase or protein such as a protein selected form the group consisting of: fibrinogen, gelatin, collagen, collagen peptides (type I, type II and type III),

It is preferable that the fixative precursor is biocompatible, and may for example be human proteins which have either been obtained from humans, or alternatively recombinantly expressed. Human fibrinogen is a preferred fixative precursor, polymerizing for instance when exposed to for instance thrombin. Suitably, the fixative may be a biocompatible medical adhesive.

In one embodiment, such as when the fixative precursor is fibrinogen, the conversion agent is thrombin or a thrombin analogue. Other coagulation factors such as Factor XIII may be added to facilitate the conversion. In a specific embodiment, ions, or salts such as sodium, calcium or magnesium, etc. that may facilitate the thrombin cleavage effect on fibrinogen rendering a polymerization may be added. Thrombin of any origin may be used, although it is preferable that a biologically compatible form is used—e.g. human recombinant thrombin may be used in the treatment of human tissue defects. Alternatively other sources of thrombin may be used, such as bovine thrombin.

Fixation may take the form of forming a gel (i.e. gelation) such as a hydrogel which locks the cells into the scaffold, whilst allowing a suitable medium for cell migration and growth, thereby facilitating the growth of new cartilage tissue through the scaffold.

In one embodiment, the biologically acceptable fixative precursor is a biologically obtained or derived component, such as fibrinogen.

The fibrinogen may be in the form of recombinant fibrinogen (e.g. recombinant human fibrinogen from HumaGene Inc., Chicago, Ill., USA). Thus, the recombinant fibrinogen may be isolated from a recombinant mammalian host cell, such as a host cell obtained or derived from the same species as the individual mammal, or a transgenic host.

Alternatively, the fibrinogen is derived and purified from blood plasma, such as human blood plasma.

Suitable concentrations of fibrinogen used include 1-100 mg/ml.

In one embodiment, particularly when the fixative precursor is fibrinogen, the conversion agent may be selected from the group consisting of: thrombin, a thrombin analogue, recombinant thrombin or a recombinant thrombin analogue.

Suitable concentrations of thrombin used are between 0.1 NIH unit and 150 NIH units, and/or a suitable level of thrombin for polymerizing 1-100 mg/ml fibrinogen.

Standard NIH units refers to the routinely used National Institute of Health standard unit for measurement of Thrombin, which according to Gaffney P J, Edgell (Thromb Haemost. 1995 September; 74(3):900-3, is equivalent to between 1.1 to 1.3 IU, preferably 1.15 IU, of thrombin.

The synthetic biodegradable scaffold, before being contacted with chondrogenic cells and before being made into a biosynthetic cartilaginous matrix, may be cut or “sized” to fit a particular defect—suitably the scaffold may be molded to a particular shape or form to suit the site of a particular defect and/or the desired shape/form of a new tissue.

The synthetic biodegradable scaffold may be any tolerated type, included but not limited to polylactic acid (PLA), polyglycolic acid (PGA) compositions.

In some embodiments the scaffold is biocompatible.

The term “biocompatible” refers to a composition or compound, which, when inserted into the body of a mammal, such as the body of patient, particularly when inserted at the site of the defect does not lead to significant toxicity or a detrimental immune response from the individual.

In one embodiment, the scaffold preferably comprises a polymer, which may be selected from the group consisting of: collagen, alginate, polylactic acid (PLA), polyglycolic acid (PGA), MPEG-PLGA or PLGA.

In one embodiment, the scaffold preferably comprises a polymer, which may be selected from the group consisting of: 1) Homo- or copolymers of: glycolide, L-lactide, DL-lactide, meso-lactide, e-caprolactone, 1,4-dioxane-2-one, d-valerolactone, R-butyrolactone, g-butyrolactone, e-decalactone, 1,4-dioxepane-2-one, 1,5-dioxepan-2-one, 1,5,8,12-tetraoxacyclotetradecane-7-14-dione, 1,5-dioxepane-2-one, 6,6-dimethyl-1,4-dioxane-2-one, and trimethylene carbonate; 2) Block-copolymers of mono- or difunctional polyethylene glycol and polymers of 1) mentioned above; 3) Block copolymers of mono- or difunctional polyalkylene glycol and polymers of 1) mentioned above; 4) Blends of the above mentioned polymers; and 5) polyanhydrides and polyorthoesters.

In some embodiments the scaffold has the ability of being hydrophilic.

It other embodiments the scaffold is porous to water and/or an isotonic buffer

In one embodiment, the scaffold essentially consists or comprises, such as comprise a majority of, a polymer, or polymers, of molecular weight, such as average molecule weight, greater than about 1 kDa, such as between about 1 kDa and about 1 million kDa, such as between 25 kDa and 75 kDa.

The scaffold or the final biosynthetic cartilaginous matrix may be in a multiple of different forms, such as a form selected from the group consisting of: a membrane, such as a porous membrane, a sheet, such as a porous sheet, an implant, a fibre, a three dimensional shape, such as a custom made implant for insertion into site of defect, a mushroom shape, a foam, a molded form, a plug, a tube, a sphere, woven or non-woven sheet, a rod, freeze dried polymer such as freeze dried polymer sheets or any combinations of these. In one particular embodiment, the shape of the scaffold or the biosynthetic cartilaginous matrix may be a disc.

Alternatively the scaffold may be a custom made three dimensional form of desired shape fitted for implantation into site of defect or site requiring implantation

Suitably, scaffolds may be of any type and size, as well as any thickness of a scaffold, such as ranging from thin membranes to several millimetres thick scaffolds.

In preferred embodiments the scaffold is synthetic.

The method of the invention may be used for cosmetic reconstruction—for example, the scaffold is made/molded into the shape required for reconstructive surgery, and the chondrogenic cells applied or fixed to the biosynthetic cartilaginous matrix with a shape suitable for the reconstruction.

The scaffold may be pre-molded to fit the exact shape of the defect, either by using the defect as a mound, or by creating the defect in a mold which is prepared using the defect as a template.

The pores of the biodegradable scaffold may be partly occupied by a component which facilitates the cell adhesion and/or in-growth for regeneration of tissue, such as a component selected from the group consisting of: chondroitin sulfate, hyaluronan, heparin sulfate, heparan sulfate, dermatan sulfate, growth factors, fibrin, fibronectin, elastin, collagen, gelatin, and aggrecan.

In one interesting embodiment, the amount of compounds which enhance cell migration and/or tissue regeneration, such as hyaluronic acid, is incorporated into the scaffold, such as at a proportion of between about 0.1 and about 15 wt %, such as between 0.1 and 10 wt %, such as such as between 0.1 and 10 wt %. In one embodiment the level is below 15 wt %, such as below 10 wt % or below 5 wt %. In one embodiment the level is above 0.01 wt % such as above 0.1 wt %, or above 1 wt %.

As discussed above the scaffolds may consist or comprise any suitable biologically acceptable material, however in a preferred embodiment the scaffold comprises of a compound selected from the group consisting of: polylactide (PLA), polycaprolacttone (PCL), polyglycolide (PGA), poly(D,L-lactide-co-glycolide) (PLGA), MPEG-PLGA (methoxypolyethyleneglycol)-poly(D,L-lactide-co-glycolide), polyhydroxyacids in general. In this respect the scaffold, excluding the pore space and any additional components, such as those which facilitates the cell adhesion and/or in-growth for regeneration of tissue, may comprise at least 50%, such as at least 60%, at least 70%, at least 80% or at least 90%, of one or more of the polymers provided herein, including mixtures of polymers.

PLGA and MPEG-PLGA are particularly preferred.

The scaffold may be prepared by freeze drying a solution comprising the compound, such as those listed above, in solution.

It is preferred that the scaffold has a porosity in the range of 20% to 99%, such as 50 to 95%, or 75% to 95%.

In one embodiment the scaffold comprises a biological polymer, i.e. a biopolymer, such as protein, polysaccharide, polyisoprenes, lignin, polyphosphate or polyhydroxyalkanoates (e.g. as described in U.S. Pat. No. 6,495,152). Suitable biopolymers may be selected from the group consisting of: gelatin, collagen, alginate, chitin, chitosan, keratin, silk, cellulose and derivatives thereof, and agarose. Other suitable biopolymers range from collagen IV to polyorganosiloxane compositions in which the surface is embedded with carbon particles, or is treated with a primary amine and optional peptide, or is co-cured with a primary amine- or carboxyl-containing silane or siloxane, (U.S. Pat. No. 4,822,741), or for example, other modified collagens (U.S. Pat. No. 6,676,969) that comprise natural cartilage material which has been subjected to defatting and other treatment, leaving the collagen II material together with glycosaminoglycans. Alternatively fibers of purified collagen II may be mixed with glycosaminoglycans and any other required additives. Such additional additives may, for example, include chondronectin or anchorin II to assist attachment of the chrondocytes to the collagen II fibers and growth factors such as cartilage inducing factor (CIF), insulin-like growth factor (IGF) and transforming growth factor (TGFβ).

The required type of scaffolds used within the context of this invention shall be scaffolds that do not act as foreign bodies in the mammal (including humans) so that no immunity or a minimum of immunity may be observed and the scaffolds used in this context shall not be toxic or significantly harmful to the organism in which it is placed. Preferably, the scaffold does not contain any microbial organisms, or any other harmful contaminants. Chondrogenic cells used in the scaffold for instance human chondrogenic cells embedded in a hydrogel, shall be capable of being placed onto the scaffold, after said scaffold is placed in its target area. The scaffold should preferably be hydrophilic so that the cell material relatively quickly is absorbed into the scaffold. However, in some instances, scaffolds may be accessible by injection with the chondrogenic cells and hydrogel. The chondrogenic cells should tolerate the scaffold with no toxic or only a minimal degree of toxicity, or no significant toxicity which may otherwise lead to detrimental results.

In one embodiment, the scaffold is in the form of a sheet, which may be pre-cut or sized to fit the defect. Such a scaffold may be, for example between 0.2 mm to 6 mm thick.

In one embodiment, the scaffold is hydrophilic, i.e. has the ability to absorb at least a small amount of water or aqueous solution (such as the cell suspension composition, e.g. the hydrogel solution), such as absorb at least 1%, such as at least such as at least 2%, such as at least 5%, such as at least 10%, such as at least 20%, such as at least 30%, such as at least 50% of the scaffold volume, of water (or equivalent aqueous solution) when placed in an aqueous solution, such as a physiological media, a buffer, or water, it is particularly beneficial that the scaffold can absorb the above amounts of the cell suspension into its porous structure.

In some embodiments, the biodegradable polymer is at least partly hydrophilic, i.e. has a component of the polymer, which may reasonable be considered hydrophilic, such as an MPEG part of an MPEG-PLGA co-polymer.

The term hydrophilic is used interchangeably with the term ‘polar’.

In the case when a non-polar scaffold is used, it is preferable that the scaffold is pretreated with an agent which facilitates the uptake of chondrogenic cells, such as a wetting agent. Wetting agents may also be used in conjunction with hydrophilic scaffolds to further improve cell penetration into the porous structure.

The biocompatible scaffold of the invention may comprise or consist of a polyester. By incorporation of a hydrophilic block in the polymer, the biocompatibility of the polymer may be improved as it improves the wetting characteristics of the material and initial cell adhesion is impaired on non-polar materials.

In a preferred embodiment the scaffold is biodegradable.

In the present context, a biodegradable polymer means a polymer that disappears over a period of time after being introduced into a biological system, which may be in vivo (such as within the human body) or, as in the present invention, in vitro (when cultured with cells); the mechanism by which it disappears may vary, it may be hydrolysed, is broken down, is biodegraded/bioresorbable/bioabsorbable, is dissolved or in other ways vanish from the biological system. When used within a clinical context this is a huge clinical advantage as there is nothing to remove from the site of repair. Thus, the newly formed tissue is not disturbed or stressed by presence of or even the removal of the temporary scaffold. It is typically preferred that the scaffold is broken down during 1 day to 10 weeks—depending on the application.

It is preferred that the scaffold is broken down prior to the clinical application at the wound or defect, but in one aspect it is regarded that a biosynthetic cartilaginous matrix with at least some polymer material remaining in the matrix may be used in vivo.

In one aspect of the invention, the scaffold is biodegradable.

As shown in the examples, it is possible to measure the biodegradability of some polymers by utilising an in vitro model—and determine the in vitro degradation of a biodegradable polymer. In one embodiment, the polymer degrades in phosphate buffer, pH 7 at 60° C., so that no more than 5% of the polymer remains after, for example 10 days, or 20 days or 30 days.

It is highly preferred that the scaffold is porous, e.g. has a porosity of at least 25%, 50%, such as in the range of 50-99.5%. Porosity may be measured by any method known in the art, such as comparing the volume of pores compared to the volume of solid scaffold. This may be done by determining the density of the scaffold as compared to a non-porous sample of the same composition as the scaffold. Alternatively Mercury Intrusion Porosimetry may be used.

In a highly interesting embodiment of the invention, the biocompatible scaffold according to the invention consists or comprises of one or more of the polymers selected form the group comprising: poly(L-lactic acid) (PLLA), poly(D/L-lactic acid) (PDLLA), Poly(caprolactone) (PCL) and poly(lactic-co-glycolic acid) (PLGA), and derivatives thereof, particularly derivatives which comprise the respective polymer backbone, with the addition of substituent groups or compositions which enhance the hydrophilic nature of the polymer e.g. MPEG or PEG. Examples are provided herein, and include a highly preferred group of polymers, MPEG-PLGA

In one embodiment, the scaffold consists or comprises a synthetic polymer.

WO 07/101,443 discloses suitable polymers for use as scaffold materials in the present invention as well as methods for their preparation.

Preferred biodegradable polymers for use in the method of the invention are composed of a polyalkylene glycol residue and one or two poly(lactic-co-glycolic acid) residue(s).

Hence, in one aspect of the for use in the method of the present invention the scaffold is prepared from, or comprises or consists of a polymer of the general formula:

A-O—(CHR¹CHR²O)ₙ—B

wherein
A is a poly(lactide-co-glycolide) residue of a molecular weight of at least 4000 g/mol, the molar ratio of (i) lactide units and (ii) glycolide units in the poly(lactide-co-glycolide) residue being in the range of 80:20 to 10:90, in particular 70:30 to 10:90, 60:40 to 40:60, such as about 50:50, such a 50:50;
B is either a poly(lactide-co-glycolide) residue as defined for A or is selected from the group consisting of hydrogen, C₁₋₆-alkyl and hydroxy protecting groups,
one of R¹ and R² within each —(CHR¹CHR²O)— unit is selected from hydrogen and methyl, and the other of R¹ and R² within the same —(CHR¹CHR²O)— unit is hydrogen,
n represents the average number of —(CHR¹CHR²O)— units within a polymer chain and is an integer in the range of 10-1000, in particular 16-250, the molar ratio of (iii) polyalkylene glycol units —(CHR¹CHR²O)— to the combined amount of (i) lactide units and (ii) glycolide units in the poly(lactide-co-glycolide) residue(s) is at the most 20:80,
and wherein the molecular weight of the copolymer is at least 10,000 g/mol, preferably at least 15,000 g/mol, or even at least 20,000 g/mol.

Hence, the polymers for use in the method of the invention can either be of the diblock-type or of the triblock-type.

In some important aspects of the invention, the synthetic biodegradable scaffold is designed to have a specific rate of degradation in vitro. This may be accomplished by varying the individual components (or ratios individual components) within the polymer.

In some embodiments the degradation time is varied by the G-L-ratio and molecular weight of MPEG-PLGA polymers: It is possible to vary the degradation time of copolymers of DL-lactide and glycolide by varying the molar ratio of lactide and glycolide. Pure polyglycolide has a degradation time of 6-12 months, poly(D,L-lactide): 12-16 months, poly(D,L-lactide-co-glycolide) 85:15 molar ratio: 2-4 months. The shortest degradation is obtained with a 50:50 molar ratio: 1-2 months. It is also possible to vary the degradation time by varying the molecular weight, but this effect is small compared to the variations possible with the L:G-ratio (see Example 4). In theory is possible to get substantially faster degradation with very low molecular weight materials, but these have mechanical properties that preclude their use for most medical devices.

In one particular embodiment A in the above formula is a poly(lactide-co-glycolide) residue of a molecular weight of at least 4000 g/mol, the molar ratio of (i) lactide units and (ii) glycolide units in the poly(lactide-co-glycolide) residue being in the range of approximately 50:50 molar ratio.

The porosity of the polymer is preferably at least 50%, such as in the range of 50-99.5%.

It is understood that the polymer for use in the method of the invention comprises either one or two residues A, i.e. poly(lactide-co-glycolide) residue(s). It is found that such residues should have a molecular weight of at least 4000 g/mol, more particularly at least 5000 g/mol, or even at least 8000 g/mol.

The poly(lactide-co-glycolide) of the polymer can be degraded under physiological conditions, e.g. in bodily fluids and in tissue. However, due to the molecular weight of these residues (and the other requirements set forth herein), it is believed that the degradation will be sufficiently slow so that materials and objects made from the polymer can fulfil their purpose before the polymer is fully degraded.

The expression “poly(lactide-co-glycolide)” encompasses a number of polymer variants, e.g. poly(random-lactide-co-glycolide), poly(DL-lactide-co-glycolide), poly(mesolactide-co-glycolide), poly(L-lactide-co-glycolide), poly(D-lactide-co-glycolide), the sequence of lactide/glycolide in the PLGA can be either random, tapered or as blocks and the lactide can be either L-lactide, DL-lactide or D-lactide.

Preferably, the poly(lactide-co-glycolide) is a poly(random-lactide-co-glycolide) or poly(tapered-lactide-co-glycolide).

Another important feature is the fact that the molar ratio of (i) lactide units and (ii) glycolide units in the poly(lactide-co-glycolide) residue(s) should be in the range of 80:20 to 10:90, in particular 70:30 to 10:90.

It has generally been observed that the best results are obtained for polymers wherein the molar ratio of (i) lactide units and (ii) glycolide units in the poly(lactide-co-glycolide) residue(s) is 70:20 or less, however fairly good results were also observed when for polymer having a respective molar ratio of up to 80:20 as long as the molar ratio of (iii) polyalkylene glycol units —(CHR¹CHR²O)— to the combined amount of (i) lactide units and (ii) glycolide units in the poly(lactide-co-glycolide) residue(s) was at the most 8:92.

As mentioned above, B is either a poly(lactide-co-glycolide) residue as defined for A or is selected from the group consisting of hydrogen, C₁₋₆-alkyl and hydroxy protecting groups.

In one embodiment, B is a poly(lactide-co-glycolide) residue as defined for A, i.e. the polymer is of the triblock-type.

In another embodiment, B is selected from the group consisting of hydrogen, C₁₋₆-alkyl and hydroxy protecting groups, i.e. the polymer is of the diblock-type.

Most typically (within this embodiment), B is C₁₋₆-alkyl, e.g. methyl, ethyl, 1-propyl, 2-propyl, 1-butyl, tert-butyl, 1-pentyl, etc., most preferably methyl. In the event where B is hydrogen, i.e. corresponding to a terminal OH group, the polymer is typically prepared using a hydroxy protecting group as B. “Hydroxy protecting groups” are groups that can be removed after the synthesis of the polymer by e.g. hydrogenolysis, hydrolysis or other suitable means without destroying the polymer, thus leaving a free hydroxyl group on the PEG-part, see, e.g. textbooks describing state-in-the-art procedures such as those described by Greene, T. W. and Wuts, P. G. M. (Protecting Groups in Organic Synthesis, third or later editions). Particularly useful examples hereof are benzyl, tetrahydropyranyl, methoxymethyl, and benzyloxycarbonyl. Such hydroxy protecting groups may be removed in order to obtain a polymer wherein B is hydrogen.

One of R¹ and R² within each —(CHR¹CHR²O)— unit is selected from hydrogen and methyl, and the other of R¹ and R² within the same —(CHR¹CHR²O)— unit is hydrogen. Hence, the —(CHR¹CHR²O)ₙ— residue may either be a polyethylene glycol, a polypropylene glycol, or a poly(ethylene glycol-co-propylene glycol). Preferably, the —(CHR¹CHR²O)ₙ— residue is a polyethylene glycol, i.e. both of R¹ and R² within each unit are hydrogen.

n represents the average number of —(CHR¹CHR²O)— units within a polymer chain and is an integer in the range of 10-1000, in particular 16-250. It should be understood that n represents the average of —(CHR¹CHR²O)— units within a pool of polymer molecules. This will be obvious for the person skilled in the art. The molecular weight of the polyalkylene glycol residue (—(CHR¹CHR²O)ₙ—) is typically in the range of 750-10,000 g/mol, e.g. 750-5,000 g/mol.

The —(CHR¹CHR²O)ₙ— residue is typically not degraded under physiological conditions, by may—on the other hand—be secreted in vivo, e.g. in from the human body.

The molar ratio of (iii) polyalkylene glycol units —(CHR¹CHR²O)— to the combined amount of (i) lactide units and (ii) glycolide units in the poly(lactide-co-glycolide) residue(s) also plays a certain role and should be at the most 20:80. More typically, the ratio is at the most 18:82, such as at the most 16:84, preferably at the most 14:86, or at the most 12:88, in particular at the most 10:90, or even at the most 8:92. Often, the ratio is in the range of 0.5:99.5 to 18:82, such as in the range of 1:99 to 16:84, preferably in the range of 1:99 to 14:86, or in the range of 1:99 to 12:88, in particular in the range of 2:98 to 10:90, or even in the range of 2:98 to 8:92.

It is believed that the molecular weight of the copolymer is not particularly relevant as long as it is at least 10,000 g/mol. Preferably, however, the molecular weight is at least 15,000 g/mol. The “molecular weight” is to be construed as the number average molecular weight of the polymer, because the skilled person will appreciate that the molecular weight of polymer molecules within a pool of polymer molecules will be represented by values distributed around the average value, e.g. represented by a Gaussian distribution. More typically, the molecular weight is in the range of 10,000-1,000,000 g/mol, such as 15,000-250,000 g/mol. or 20,000-200,000 g/mol. Particularly interesting polymers are found to be those having a molecular weight of at least 20,000 g/mol, such as at least 30,000 g/mol.

The polymer structure may be illustrated as follows (where R is selected from hydrogen, C₁₋₆-alkyl and hydroxy protecting groups; n is as defined above, and m, p and ran are selected so that the above-mentioned provisions for the poly(lactide-co-glycolide) residue(s) are fulfilled):

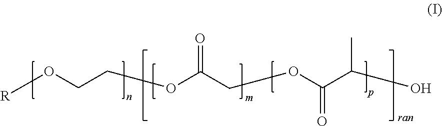

### Diblock-Type Polymer

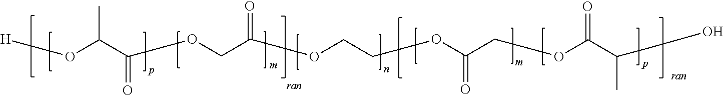

### Triblock-Type Polymer

For each of the above-mentioned polymer structures (I) and (II) will be appreciated that the lactide and glycolide units represented by p and m may be randomly distributed depending on the starting materials and the reaction conditions.

Also, it is appreciated that the lactide units may be either D/L or L or D, typically D/L or L.

As mentioned above, the poly(lactide-co-glycolide) residue(s), i.e. the polyester residue(s), is/are degraded hydrolytically in physiological environments, and the polyalkylene glycol residue is secreted from, e.g. the mammalian body. The biodegradability can be assessed as outlined in the Experimentals section.

The polymers can in principle be prepared following principles known to the person skilled in the art.

In principle, polymer where B is not a residue A (diblock-type polymers) can be prepared as follows:

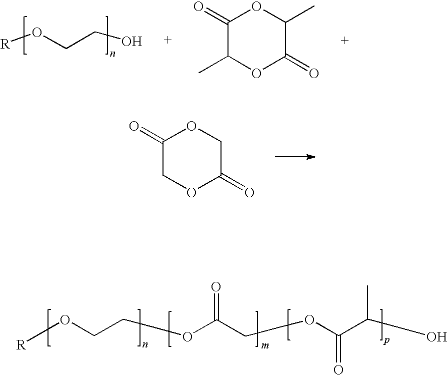

In principle, polymer where B is a residue A (triblock-type polymers) can be prepared as follows:

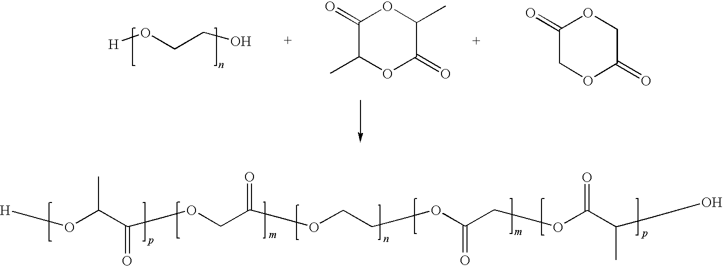

Unless special conditions are applied, the distribution of lactide units and glycolide units will be randomly distributed or tapered within each poly(lactide-co-glycolide) residue.

Preferably the ratio of glycolide units and lactide units present in the polymer used in scaffold is between an upper limit of about 80:20, and a lower limit of about 10:90, and a more preferable range of about 60:40 to 40:60.

Preferably the upper limit of PEG-content is at most about 20 molar %, such as at most about 15 molar %, such as between 1-15 molar %, preferably between 4-9 molar %, such as about 6 molar %.

The synthesis of the polymers is illustrated in WO 2007/101443.

The scaffold may, e.g. be a biodegradable, porous material comprising a polymer as defined herein, wherein the porosity is at least 50%, such as in the range of 50-99%.

The high degree of porosity can be obtained by freeze-drying.

The void space of the material of the polymer may be unoccupied so as to allow or even facilitate cell adhesion and/or in-growth into the synthetic biodegradable scaffold. In one embodiment, the pores of the material are at least partly occupied by a component from the extracellular matrix. Examples of components from the extracellular matrix are chondroitin sulfate, hyaluronan, hyaluronic acid, heparin sulfate, heparan sulfate, dermatan sulfate, growth factors, fibrin, fibronectin, elastin, collagen, gelatin, and aggrecan.

As discussed elsewhere, the scaffold may also contain the conversion agent thrombin either alone or in combination with one of the above mentioned.

The components from the extracellular matrix could be added either as particles, which are heterogeneously dispersed, or as a surface coating. The concentration of the components from the extracellular matrix relative to the synthetic polymer is typically in the range of 0.5-70% (w/w), such as 3-70%, preferably 30-50%. In another aspect, the concentration is below 10% (w/w). Moreover, the concentration of the components of the extracellular matrix is preferably at the most 0.3% (w/v), e.g. at the most 0.2 (w/v), relative to the volume of the material.

The porous materials may be prepared according to known techniques, e.g. as disclosed in Antonios G. Mikos, Amy J. Thorsen, Lisa A Cherwonka, Yuan Bao & Robert Langer. Preparation and characterization of poly(L-lactide) foams foams. Polymer 35, 1068-1077 (1994). One very useful technique for the preparation of the porous materials is, however, freeze-drying.

In one embodiment, the synthetic biodegradable scaffold is a scaffold as prepared by the method disclosed in WO 07/101,443. The method is particularly suited to prepare scaffolds from PLGA and MPEG-PLGA polymers.

In some aspects of the present invention, the synthetic biodegradable scaffold is a scaffold prepared by the method disclosed in WO 07/101,443, which method comprises the steps of:
(a) dissolving a polymer as defined herein in a non-aqueous solvent so as to obtain a polymer solution;
(b) freezing the solution obtained in step (a) so as to obtain a frozen polymer solution; and
(c) freeze-drying the frozen polymer solution obtained in step (b) so as to obtain the biodegradable, porous material.

The non-aqueous solvent used in the method as disclosed in WO 07/101,443 should with respect to melting point be selected so that it can be suitable frozen. Illustrative examples hereof are dioxane (mp. 12° C.) and dimethylcarbonate (mp. 4° C.).

In one variant of the method as disclosed in WO 07/101,443, the polymer solution, after step (a) above is poured or cast into a suitable mould. In this way, it is possible to obtain a three-dimensional shape of the material specifically designed for the particular application.

In embodiments, wherein particles of components from the extracellular matrix is used in the methods according to the invention, these extracellular matrix components may be dispersed in the solution obtained in step (a) before the solution (dispersion) is frozen at defined in step (b).

The components from the extracellular matrix may, for instance, be suspended in a suitable solvent and then added to the solution obtained in step (a). By mixing with the solvent of step (a), i.e. a solvent for the polymer defined herein.

In one aspect, the biodegradable, porous material obtained in step (c), in a subsequent step, is immersed in a solution of glucosaminoglycan (e.g. hyaluronan) and subsequently freeze-dried again.

In some alternative embodiments, the material are present in the form of a fibre or a fibrous structure prepared from the polymer defined herein, possibly in combination with components from the extracellular matrix. Fibres or fibrous materials may be prepared by techniques known to the person skilled in the art, e.g. by melt spinning, electrospinning, extrusion, etc. Such fibers are disclosed in WO 2007/122232.

In some embodiments, the synthetic biodegradable scaffold is biocompatible. Even if the scaffold structure according to the invention is degraded, scaffold degradation products may still be present in the biosynthetic cartilaginous matrix. Accordingly, it may still be an advantage to use biocompatible scaffold material.

In some embodiments, the synthetic biodegradable scaffold is part of a component which further comprises a biopolymer, such as a non-synthetic biopolymer, such as polysaccharides, polypeptides, lignin, polyphosphate or polyhydroxyalkanoates. In some embodiments this biopolymer is selected from the group consisting of: gelatin, hyaluronan, hyaluronic acid (HA), dermatan sulphate, collagen, such as collagen type I and/or type II, alginate, chitin, chitosan, keratin, silk, cellulose and derivatives thereof, and agarose.

In some embodiments, the synthetic biodegradable scaffold is part of a component which further comprises a biopolymer of any suitable extracellular matrix component.

### EXAMPLES

### Example 1

### In Vitro Study of Chondrogenesis of hAC-Loaded MPEG-PLGA Scaffolds

These in vitro studies were done in order to evaluate the degree of chondrogenic matrix synthesis in a 3-dimensional scaffold system base on a polymer part and a cellular/hydrogel part. The results from this study will indicate whether the tested scaffold system could be a candidate in an in vivo cartilage repair study.

The scaffold system tested in this in vitro study is composed of three major parts:
1. The polymer part: MPEG-PLGA (methoxypolyetheleneglycol-block-poly(lactide-co-glycolide)), Coloplast NS.
2. The cellular part; human articular chondrocytes (hACs)
3. The hydrogel part; Fibrin-based gel.

The MPEG-PLGA polymer is able to absorb liquid due to its hydrophillic characteristics and in this way a cellular suspension can be distributed into the scaffold structure. The cellular part used for this in vitro study is normal hACs of low passages, which is an important parameter affecting the degree of matrix synthesis in the system. hACs of low passages does not demonstrate the same extensive signs of dedifferentiation as hACs of higher passages.

### Materials

Chemicals, general: Dulbecco's-modified Eagle's Medium (DMEM:F12)+GlutaMAX-1, (GIBCO), Fungizone, (GIBCO), Gentamicin, (GIBCO), Phosphate-buffered saline, (PBS), Trypsin/EDTA, (GIBCO), Fetal Bovine Serum, batch tested, (FBS), (Cambrex), Fibrinogen, (Sigma), Thrombin, (Sigma), CaCl2, (Sigma)

### Chemicals, Analysis:

Histology: Toluidine Blue O, (Sigma), Saranine O, (Sigma), Immunohistochemistry (IHC): Dako REAL™ Detection System Peroxidase/DAB+, (DAKO)

TABLE 1
Antibodies

| Name | Antigen | Clone | Manufacturer |
|---|---|---|---|
| Anti-Collagen Type | Collagen Type II | II-4AC11 | Calbiochem |
| II (Ab-1) Mouse | | | |
| mAb (II-4AC11) | | | |
| Mouse Monoclonal | Proteoglycan | 1R11 14A6 | AH Diagnostic |
| Anti-Human | | | |
| Proteoglycan | | | |
| (AHP0012) | | | |

Molecular analysis: RNAgents® Total RNA Isolation System, (Promega), PCR Master Mix, (Promega), AMV Reverse Transciptase, (Promega), RNALater, (Sigma)

TABLE 2
Primers (produced by TAG Copenhagen A/S)

| | | | Annealing |
|---|---|---|---|
| | | | temper- |
| | | | ature |
| Gene | Primer sequence (5′-3′) | | (° C.) |
| GAPDH* | Sense: | GGGCTGCTTTTAACTCTGGT | 55 |
| | Antisense: | GCAGGTTTTTCTAGACGG | |
| Aggrecan | Sense: | TGAGGAGGGCTGGAACAAGT | 56 |
| | | ACC | |
| | Antisense: | GGAGGTGGTAATTGCAGGGA | |
| | | ACA | |
| Col(II)** | Sense: | GGACACAATGGATTGCAAGG | 55 |
| | Antisense: | TAACCACTGCTCCACTCTGG | |
| Sox9 | Sense: | ATCTGAAGAAGGAGAGCGAG | 55 |
| | Antisense: | TCAGAAGTCTCCAGAGCTTG | |

*Glyceralaldehyde-3-phosphate dehydrogenase
**Collagen Type II

Plastic: Multidishes (12 wells, polystyrene), NUNC. Tissue culture flasks (80 cm2, polystyrene), NUNC, Tissue culture flasks (75 cm2, polystyrene), NUNC.

### Procedure

Harvest of hACs and Combining them with Fibrinogen Solution

hAC cultures of passage 1-3, reaching 70-80% confluence (within 80 cm2 tissue culture flasks) were used in this study. After washing the growth medium (DMEM/F12 containing FBS [16%], ascorbic acid, gentamicin, fungizone) out of the culture flask with PBS, trypsin/EDTA (5 ml/80 cm2 flask) was added in order to release the cells form the surface. After 5 min incubation with trypsin/EDTA, 10 mL growth medium was added and the cells were centrifuged for 10 min at 1100 rpm. Subsequently the supernatant was discarded and the pellet was resuspended with 5 mL growth medium. Fibrinogen was solubilized (50 mg/mL) in DMEM/F12 at 37° C. for 1 hour. After totally dissolving the fibrinogen the solution was filter sterilized through a 0.2 μm filter. hACs were resuspended in 1 mL fibrinogen solution (10×106 hACs/mL).

Loading hACs/Hydrogel on MPEG-PLGA Scaffolds

MPEG-PLGA scaffolds (1 cm2) were placed in 12 well multidishes and 100 μL fibrinogen/hAC solution was added on top of each scaffold together with a thrombin/CaCl₂ solution. After 5 min each 2 mL growth medium was added to each well. Multidishes were placed in a humidified atmosphere of 5% CO₂ at 37° C.

### Processing of Cultured Scaffolds for Analysis

After 3, 6 and 12 weeks the MPEG-PLGA scaffolds were processed for subsequent analysis. Each scaffold was divided into two parts; one part was placed in formalin at 4° C. and the other part was placed in RNALater solution in order preserve the RNA present within the scaffold structure. Samples for RNA purification were stored at −20° C.

### Processing of Cultured Scaffolds for Migration Analysis

After 4 weeks the MPEG-PLGA scaffolds were processed for migration assay. The centre of the scaffolds was removed and examined under light microscopy in order to remove possible hACs adhering to the scaffold structure. The scaffold explants were placed in 25 cm² tissue culture flasks containing 14 mL growth medium. The migration of hACs out of the scaffold explants was carefully observed under light microscopy and compared with the migration out of human articular cartilage explants.

### RT-PCR Analysis

Total cellular RNA was isolated using a commercially available RNA isolation kit, in accordance with the manufacturer's instruction. Purity of RNA was confirmed by measuring the absorbance at 260 nm and 280 nm and calculating the 260/280 ratio. RNA was eluted in RNase-free water and stored at −80° C. until further use. First-strand complementary DNA (cDNA) was synthesized from 1 μg RNA by using. By using the same amount of RNA, final cell number did not affect the PCR analysis. cDNA synthesis was performed by reverse transcription in a reaction mixture containing AMV Reverse Transciptase. PCR reactions (25 μL) were set up using Taq DNA polymerase and run on a thermocycler (Techne TC-312) with an initial denaturation step at 95° C. for 5 min subjected to 30 cycles of PCR (95° C. for 1 min, specific annealing temperature for 30 sec, 72° C. for 1 min) followed by a final extension at 72° C. for 7 min. For each PCR amplification, an aliquot of each product was electrophoresed in 1% agarose gel. The gel was stained with 0.8 μg/mL ethidium bromide and photographed. All reactions included negative controls without template.

### Histology

Scaffolds were embedded in paraffin, sectioned and stained with hematoxylin and eosin stain (H&E) at Bangs Laboratory, Fredericiagade 33, 1310 Copenhagen. Sections were deparafinized and stained with 0.5% Toluidine Blue 0 or 0.5% Safranin 0 for 10 min and subsequently washed with tap water. For immunohistochemistry analysis, sections were deparafinized and then treated with 3% H₂O₂ for 15 min. Subsequently sections were blocked for 10 min with goat serum and then stained with the primary antibodies listed in table 1 overnight at 4° C. (final antibody concentrations are listed in table 3). The antigen presence was evaluated with Dako REAL™ Detection System Peroxidase/DAB+. Stained sections were analysed under light microscopy and microphotographs were taken when appropriate.

TABLE 3
(Antibody concentrations)

Antibody

| | Anti Col(II) | Anti aggrecan |
|---|---|---|
| Final concentration | 10 μg/mL | 1:80 |

### Results

### Histology

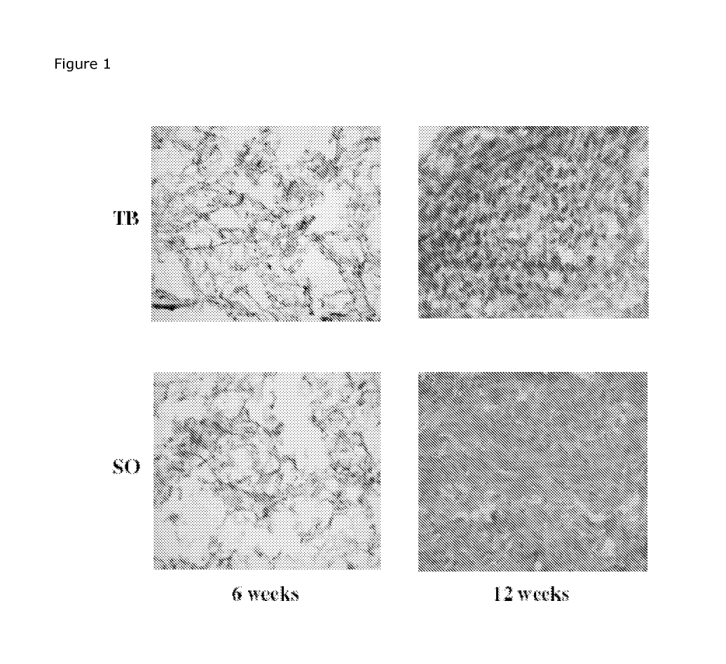

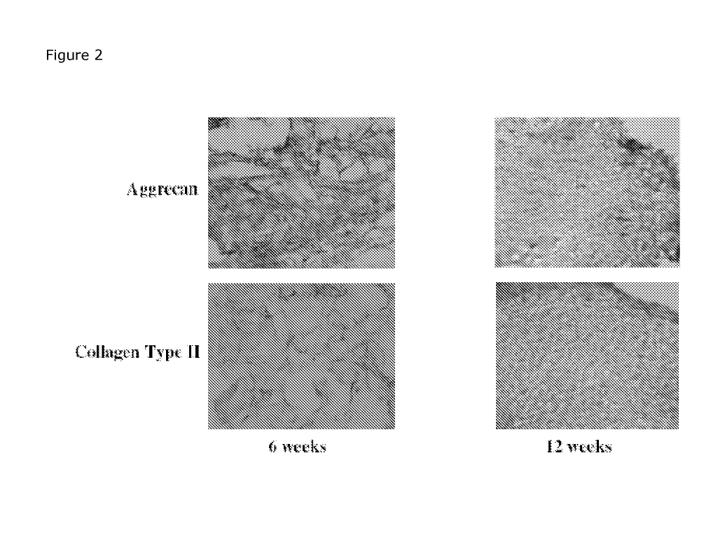

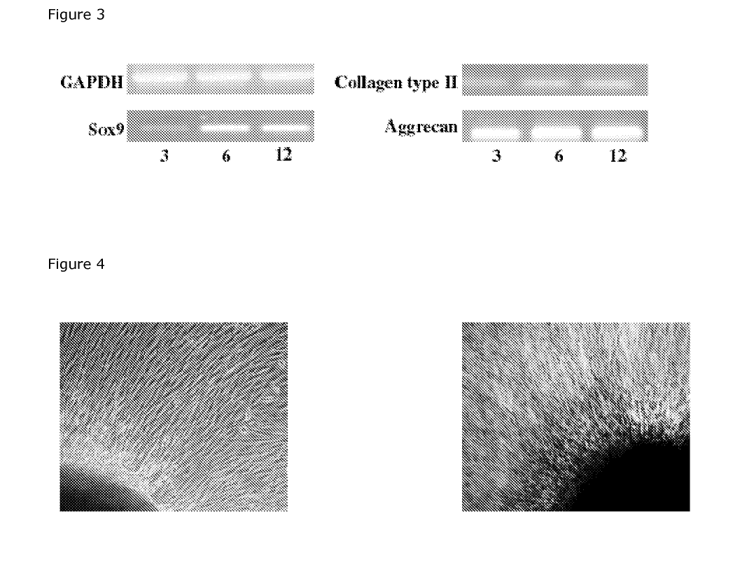

FIG. 1 shows a representative microphotographs of hAC-loaded MPEG-PLGA scaffolds after staining.

Staining sections with TB and SO, demonstrated that hACs are able to adhere and lay down chondrogenic extracellular matrix components within a MPEG-PLGA scaffold, studied under specific in vitro conditions.

The IHC analysis confirmed the findings with TB and SO, and demonstrated that the essential chondrogenic markers for normal articular cartilage tissue are present within the scaffold structure after culture.

### RT-PCR Analysis

In the RT-PCR analysis an upregulation of the collagen type II and aggrecan was observed depending on time in culture. Furthermore the transcription factor necessary for driving and maintaining the hACs in the chondrocyte lineage was present and furthermore upregulated.

### Migration Analysis

FIG. 4 illustrates the migration out of the central part of the MPEG-PLGA scaffold system, observed after 5 days.

The migration analysis demonstrated that hACs residing in the centre of the MPEGPLGA scaffold, a location, where the nutrient supply could be critical, were able to divide and migrate out from the scaffold structure. The migration pattern was comparable to normal articular cartilage explants.

### Conclusion

The in vitro study demonstrated that the MPEG-PLGA scaffold system supports the synthesis of essential chondrogenic matrix proteins and that the microenviroment ensures that hACs lay down these molecules. Furthermore the scaffold components are not toxic to hACs and do not inhibit the migration. In conclusion the MPEG-PLGA scaffold can be used for an in vivo experiment, evaluating the cartilage repair potential of such a system.

### Example 2

### Degradation of Scaffolds of MPEG-PLGA 2.000-30.000 and EDC Cross-Linked Gelatine in Wound Exudate, 10% FCS in DMEM, FCS and PBS. A 14-Day Study

The present example demonstrates the degradation of MPEG-PLGA and EDC cross-linked gelatine, when incubated in wound exudates, medium, serum and PBS at 37° C. for up to 14 days.

### Material and Methods

Eight mm biopsies were punched out of MPEG-PLGA and EDC cross-linked gelatine (150506E) scaffold and placed in it's own well in a 48 well plate. The scaffolds were covered by 1 ml of respectively wound exudates (debrided ischemic diabetic leg ulcer with low elastase level, collected using VAC therapy, properly corresponding to acute wound exudates), medium (10% Fetal Calf Serum (FCS) in Dulbecco's Modified Eagle's Medium (DMEM)), FCS and PBS pH 7.4. The scaffolds were tested in duplicates.

The plates were incubated for 1, 3, 8 and 14 days at 37° C. RH 50% after which the scaffolds were placed on a glass plate and photographed.

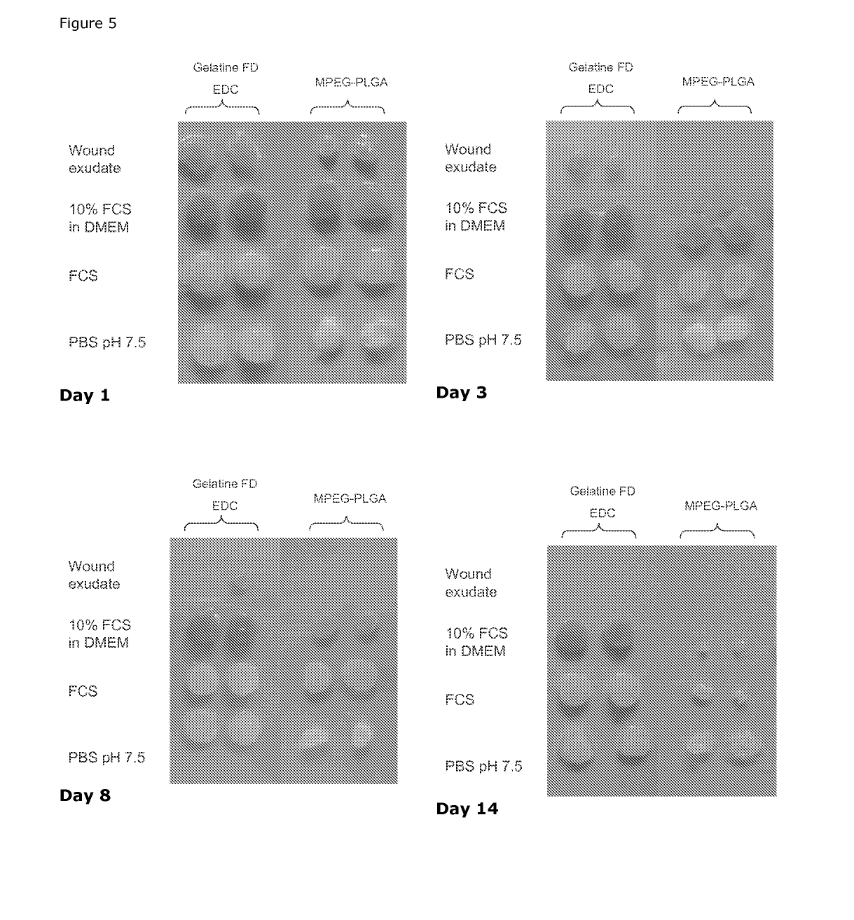

Result and Conclusion as shown in FIG. 5. When MPEG-PLGA scaffold was incubated in wound exudates the scaffold diminished considerable in size already at day 1 and was totally degraded at day 3. When the scaffold was incubated in medium, FCS or PBS the first apparent reduction in size were at day 8 and at day 14 there was an obvious larger reduction in size when MPEG-PLGA was incubated in medium or FCS compared to PBS.

EDC cross-linked gelatine showed also a considerable reduction in size when incubated in wound exudates but first at day 3. At day 8 only one of the duplicates was totally degraded but at day 14 no scaffold were left. Incubation in medium, FCS or PBS did not change the size of the scaffolds at any time during the study.

In conclusion, MPEG-PLGA scaffold is degraded faster than EDC cross-linked gelatine scaffold with wound exudates being the most effective incubation solution.

### Example 3

### Determination of Remaining Scaffold Material in In Vitro Cultured Cartilage

Preparation of Scaffolds of Mpeg-Plga: Metoxy-Polyethylene Glycol—Poly(Lactide-Co-glycolide) (Mn 2.000-30.000, L:G 1:1) are dissolved in 1,4-dioxane to solutions containing 4%. Ten ml of the solution are poured into a 7.3×7.3 cm mould and frozen at −5° C. and lyophilised at −20° C. for 5 h and 20° C. for approx 15 h. The samples should afterwards be placed in draw (hydraulic pump) in desiccators for 24 h.

Scaffolds of MPEG-PLGA will be cultivated with chondocytes to produce in vitro cartilage as described previously. After cultivation will the scaffolds be placed in Lillys fixative for 3 days before the scaffolds are embedded in paraffin and sectioning into 8 μm slices.

An appropriate histological staining technique like Meyer's haematoxylin erosion (HE), Masson's trichrome or similar will be used to stain the new tissue but not the scaffold material. Digital images (10× and 20× magnifications) will be taken as composite pictures using a BX-60 Olympus microscope fitted with a Prior Optiscan xy-table (ES110EXT, Prior Scientific Instruments Ltd.) and an Evolution MP cooled colour camera (Media Cybernetics). Each sample will be tested in three and 5-10 slices made of each. Digital image will be taken of all made slices and the amount of remaining scaffold material calculated using Image Pro Plus 5.1 software e.g. remaining scaffold material as % of total scaffold.

Growth of Fibroblast and Smooth Muscle Cells Together with Particles of MPEG-PLGA

Attachment and growth of fibroblasts and smooth muscle cells on particles of MPEG-PLGA will be tested with the particles placed in the bottom of the culture well or in suspension together with fibroblast or smooth muscle cells in low attachment culture plate to prevent the cells from adhering to culture well.

### Particles Placed in the Bottom of the Culture Well.

Particles of MPEG-PLGA will be suspended in an appropriate solvent e.g. 99% ethanol or likewise. The particles should be in a low concentration to keep the particles separated to prevent clotting. Different volumes of the suspension will be measured into wells in 12 well culture plates. The culture plates will be placed in a sterile hood to evaporate the solvent.

Primary human fibroblasts or smooth muscle cells will be seeded on top of the particles with densities between 1×10³/cm² and 1×10⁵/cm². The cells will be applied in a small volume of growth medium and incubated at 37° C. at 5% CO₂ before additional growth medium will be added. Evaluation of the cells attachment, morphology, growth and population of the particles will be preformed at appropriate time e.g. day 1, 3 and 7 by staining the cells with neutral red followed by evaluation using an Leica DMIRE2 inverted microscope fitted with a Evolution MP cooled colour camera (Media Cybernetics). Digital images will be taken using Image Pro Plus 5.1 software (Media Cybernetics). The number of cells adhering to the particles will be calculated by using Cytotoxicity Detection Kit (LDH, Roche Diagnostics GmbH) or 3-(4,5-dimethylthiazol-2-yl)-2,5-diphenyl tetrazolium bromid (MTT, Sigma M-2128).

### Particles and Cells in Suspension in Low Cell Attachment-Plates.

In culture wells coated with poly(2-hydroxyethyl methacrylate) (poly-HEMA) or likewise, will different concentrations of fibroblasts or smooth muscle cells be mixed together with particles of MPEG-PLGA. Evaluation of the cells attachment, morphology, growth and population of the particles will be preformed at appropriate time e.g. day 1, 3 and 7 by staining the cells with neutral red followed by evaluation using an Leica DMIRE2 inverted microscope fitted with a Evolution MP cooled colour camera (Media Cybernetics). Digital images will be taken using Image Pro Plus 5.1 software (Media Cybernetics). The number of cells adhering to the particles will be calculated by using Cytotoxicity Detection Kit (LDH, Roche Diagnostics GmbH) or 3-(4,5-dimethylthiazol-2-yl)-2,5-diphenyl tetrazolium bromid (MTT, Sigma M-2128).

### Example 4

### Accelerated Degradation Study of MPEG-PLGA 2-30

An accelerated degradation study of MPEG-PLGA 2-30 in phosphate buffer at 60° C. shows complete degradation after 10 days. This corresponds to 50 days at 37° C.

Materials and methods:
Scaffolds (MPEG-PLGA 2-30 with a 50:50 DL-lactide to glycolide ratio).
12 ml screw-cap vials
GPC
Buffer: 7.4 g Na₂HPO₄+2.15 g KH₂PO₄ is dissolved in 900 mL water. pH is adjusted to 7.0 using diluted H₃PO₄ and volume adjusted to 1 L.
Approx. 4 mg scaffold is weighed to a vial (×5), and 3 ml buffer is added. The vials are placed in an oven at 60° C. and a vial is removed at 3, 4, 5, 6 and 10 days (vials are placed in the freezer until further work).
The vials are freeze dried at −5° C. overnight, dried in a vacuum dessicator overnight, dissolved in 2 mL THF:DMF 1:1, filtered and analyzed on the GPC.

### Results:

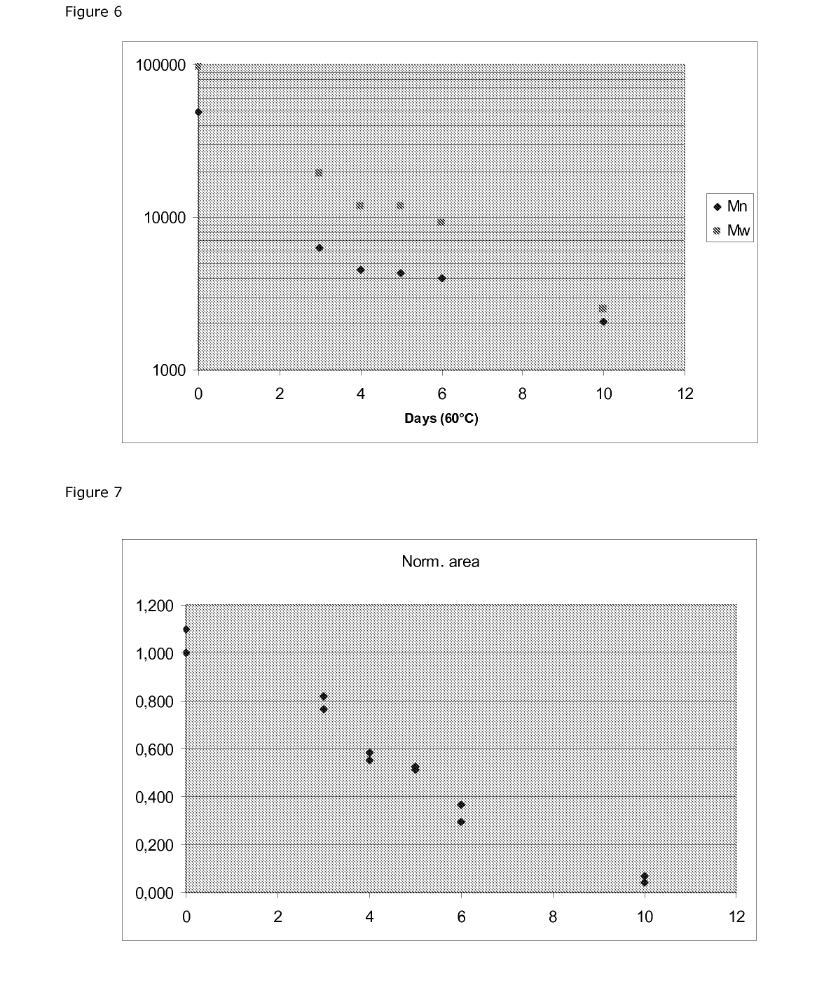

The results are illustrated graphically in FIGS. 6 and 7.

| Days | Weight | | | | | Mw | Area | Area |
|---|---|---|---|---|---|---|---|---|
| (60° C.) | (mg) | Mn | Mw | RI area | Mn (avg) | (avg) | (avg) | (norm.) |
| 0 | 5.61 | 50421 | 96460 | 16.55 | | | | 1.000 |
| 0 | | 47678 | 96218 | 18.19 | 49049 | 96339 | 17.37 | 1.099 |
| 3 | 4.39 | 5872 | 19190 | 13.22 | | | | 0.817 |
| 3 | | 6669 | 19769 | 12.38 | 6270 | 19479 | 12.8 | 0.765 |
| 4 | 4.43 | 4466 | 12103 | 9.5 | | | | 0.582 |
| 4 | | 4549 | 11876 | 8.99 | 4507 | 11989 | 9.245 | 0.550 |
| 5 | 4.13 | 4388 | 11902 | 8.02 | | | | 0.527 |
| 5 | | 4274 | 11965 | 7.79 | 4331 | 11933 | 7.905 | 0.511 |
| 6 | 3.87 | 3517 | 8875 | 5.21 | | | | 0.365 |
| 6 | | 4460 | 9609 | 4.16 | 3988 | 9242 | 4.685 | 0.291 |
| 10 | 4.67 | 1973 | 2477 | 1.16 | | | | 0.067 |
| 10 | | 2184 | 2512 | 0.71 | 2078 | 2494 | 0.935 | 0.041 |

After 10 days, complete degradation is seen, and the only peak remaining in the chromatogram is MPEG. This would correspond to approximately 50 days at 37° C.

### Conclusion:

A method for the rapid determination of the degradation rate of PLGA in vitro has been developed. A minimum of sample preparation is required. Complete degradation of a 2-30 MPEG-PLGA scaffold (4%) is seen after 10 days/60° C.

### Example 5

### Cell-Seeding

This example describes the preparation of a tissue engineered cartilage matrix suitable for decellularization.

Human articular chondrocytes (hACs) are obtained by explant culture from human cartilage biopsies. hACs are cultured in a medium containing DMEM/F12, 16% fetal bovine serum (FBS), ascorbic acid (75 μg/ml), fungizone (2.4 μg/ml) and gentamicin (10 mg/ml). When a confluence level of 80% is reached hACs are trypsinized and seeded evenly onto the biopolymer at a concentration of 50×10⁶ hACs/cm³. Cells are allowed to attach to the biopolymer for 1 hour and then fresh medium is added. The engineered cartilage is cultured for 8 weeks in a dynamic culture system in atmosphere of 5% CO₂ at a temperature of 37° C.

### Example 6

### Decellularization (Removal of Antigens Derived from Chondrogenic Cells and/or Complete Removal of Chondrogenic Cells)

The following method describes a process for removing the entire antigenic content, preserving the three-dimensional architecture of the extracellular matrix, of a biosynthetic cartilaginous matrix.

The biosynthetic cartilaginous matrix containing chondrogenic cells is transferred to a 50 ml sterile, screw capped tube and incubated in a hypotonic solution consisting of 12 mM TRIS pH 8.0 (ACS grade, for cell culture), 5 mM EDTA, supplemented with 0.1 mM Butylated hydroxyanisole (BHA, Sigma B-1253 or equivalent) and 0.1 μM PMSF. The incubation period is 14 hours, at 4° C., on a shaking platform. Subsequently the biosynthetic cartilaginous matrix are placed in a 8 mM 3-[(3-cholamidopropyl)dimethylammonio]-1-propanesulfate (CHAPES) for 1 hour at room temperature. Then the biosynthetic cartilaginous matrix is rinsed extensively in PBS without Ca²⁺ and Mg²⁺ to remove residual solution.

Alternatively de-cellularization is accomplished by the use of a non-ionic detergent method be applying the biosynthetic cartilaginous matrix to a de-cellularization solution containing Triton X-100, EDTA, RNAse, and DNAse.

### Example 7

### Demonstration of the Removal of Cellular Material from the Cartilaginous Matrix

The prepared biosynthetic cartilaginous matrix is paraffin-embedded and sections are made of a thickness of 10 μm. Before subsequent analysis, sections are deparafinized by a first incubation for 10 min in xylene, and then hydrated through graded alcohols (70, 90, 100% ethanol).

Two different analyses are used in order to demonstrate the removal of cellular materials from the cartilaginous matrix. In the first analysis hydrated sections are washed twice in phosphate buffered saline (PBS) and then stained for 10 min in hematoxylin and eosin in order to determine if any nuclear structures can be observed.

After staining sections are dried and mounted with coverslips using Pertex.

In the second analysis inspection for the presence of DNA is performed. Hydrated sections are washed twice in PBS and then stained with 1 μg/mL DAPI in PBS for 1 min. Subsequently the sections are washed 3 times with 0.1% Triton X-100 in PBS.

The following light microscopy analysis should not reveal any signs of cellular materials within the cartilaginous matrix based on the analysis for nuclear structures and the presence of DNA.

### Example 8

### Visualizing the Presence of Extracellular Molecules

The prepared biosynthetic cartilaginous matrix is paraffin-embedded and sections are made of a thickness of 10 μm. Before subsequent analysis, sections are deparafinized by a first incubation for 10 min in xylene, and then hydrated through graded alcohols (70, 90, 100% ethanol).

Two different analyses are used in order to demonstrate the presence of extracellular matrix proteins within the cartilaginous matrix.

In the first analysis hydrated sections are washed twice in phosphate buffered saline (PBS) and then stained for 10 min in 0.5% safranin 0 in order to determine if any glycosaminoglycans (GAGs) are present. After staining sections are rinsed in tap water and mounted with coverslips using Pertex.

In the second analysis hydrated sections are washed twice in PBS and then incubated for 15 min in 0.1% H₂O₂ to quench endogenous peroxidase activity. Sections are then washed twice in PBS and placed in Antigen Retrieval Solution (Dako) in a microwave. After heat-treatment in the microwave sections are equilibrated to room temperature and subsequently washed three times in distillated water.

Monoclonal antibodies against human aggrecan and human collagen type II (both purchased from Santa Cruz Biotechnology) are applied to the sections at a concentration of 5 μg/ml and 1:80 dilution respectively. Actual presence of the two extracellular molecules are visualized by ChemMate System (Dako).

Both analyses will demonstrate the presence of extracellular matrix proteins like GAGs, aggrecan and collagen type II.

### Example 9

### Visualizing the Presence of DNA/RNA within the Biosynthetic Cartilagenius Matrix by PCR

RNA is extracted from the decellularized matrix by Total RNA Isolation (Promega) and cDNA is synthesized by RT-System (Promega). The expression of the house-keeping gene Glyceralaldehyde-3-phosphate dehydrogenase (GAPDH), is analysed by PCR using specific primers for GAPDH; Sense: 5′GGGCTGCTTTTAACTCTGGT 3′ and Antisense: 5′GCAGGTTTTTCTAGACGG3′ (DNA, Technology, Copenhagen).

After amplification agarose gels are quantitavely analysed by AlphaImager (Alpha Innotech, CA).

The analysis should reveal no expression of GAPDH within the decellularized matrix, demonstrating that no cellular DNA/RNA will remain in structure after decellularization.

The expression of chondrogenic markers like collagen type II and aggrecan may also be analysed by PCR using specific primers for these markers.

The lack of expression in the AlphaImager analyses will support the results obtained with the GAPDH analysis.

1. A method for the preparation of a biosynthetic cartilaginous matrix suitable of implantation into a living individual mammal, such as a human being, said method comprising the sequential steps of:
a) contacting in vitro a population of chondrogenic cells with a synthetic biodegradable scaffold;
b) culturing in vitro for a period of time said chondrogenic cells within said synthetic biodegradable scaffold so that the chondrogenic cells produce a biosynthetic cartilaginous matrix;
c) substantially removing any antigen derived from said chondrogenic cells;
wherein during any one of steps a)-c) and/or in a subsequent step the biodegradable scaffold is completely or partially degraded in vitro.

2. The method according to claim 1, wherein the synthetic biodegradable scaffold is sterilised prior to step a) through the application of irradiation, such as beta radiation, or plasma sterilisation.

3. The method according to claim 1, wherein the synthetic biodegradable scaffold is completely or partially degraded by free radical degradation.

4. The method according to claim 1, wherein the synthetic biodegradable scaffold is completely or partially degraded by cellular degradation.

5. The method according to claim 1, wherein step c) is performed by substantially removing said population of chondrogenic cells, from said biosynthetic cartilaginous matrix.

6. The method according to claim 1, wherein step a) and/or step b) further comprises administering a component which facilitates the cell adhesion and/or in-growth for generation of biosynthetic cartilaginous matrix within the synthetic biodegradable scaffold, such as a component selected from the group consisting of: chondroitin sulfate, hyaluronan, hyaluronic acid (HA), heparin sulfate, heparan sulfate, dermatan sulfate, growth factors, fibrin; fibronectin, elastin, collagen, such as collagen type I and/or type II, gelatin, and aggrecan, or any other suitable extracellular matrix component.

7. The method according to claim 1, wherein step a) and/or step b) further comprises administering a suspension of extracellular matrix components produced by a chondrogenic cells.

8. The method according to claim 1, wherein step a) and/or step b) further comprises administering a further compound to the synthetic biodegradable scaffold, wherein said further compound is selected from the group consisting of: growth factors, such as Insulin-like growth factor 1 (IGF-1), or transforming growth factors (TGFs), such as TGF-alpha or TGF-beta, or FGFs, such as FGF-1 or FGF-2.

9. The method according to claim 1, wherein hyaluronic acid is incorporated into said synthetic biodegradable scaffold.

10. The method according to claim 9, wherein the hyaluronic acid is present in said synthetic biodegradable scaffold at a proportion of between about 0.1 and about 15 wt %.

11. The method according to claim 1, wherein dermatan sulphate is incorporated into said synthetic biodegradable scaffold.

12. The method according to claim 11, wherein the dermatan sulphate is present in said synthetic biodegradable scaffold at a proportion of between about 0.1 and about 15 wt %.

13. The method according to claim 1, wherein said population of chondrogenic cells is selected from the list consisting of chondrocytes, such as human articular chondrocytes, stem cells or equivalent cells capable of transformation into a chondrocyte, such as mesenchymal stem cells or embryonic stem cells.

14. The method according to claim 1, wherein said chondrogenic cells are non-autologous and/or non-homologous relative to the living individual mammal, wherein the cartilaginous matrix is implantated.

15. The method according to claim 1, wherein said chondrogenic cells are in the form of a cell suspension, cell associated matrix, or tissue explant.

16. The method according to claim 1, wherein said chondrogenic cells are introduced under step a) in an amount of about 0.1×10⁴ cells to about 10×10⁶ cells per 0.1 cm³ of synthetic biodegradable scaffold.

17. The method according to claim 1, wherein said chondrogenic cells are cultured under step (b) for a period of at least 1 week, such as at least 2 weeks, such as at least 3 weeks, such as at least 6 weeks, such as at least 12 weeks.

18. The method according to claim 1, wherein said synthetic biodegradable scaffold is porous to water and/or an isotonic buffer.

19. The method according to claim 1, wherein said synthetic biodegradable scaffold essentially consists or comprises a polymer of molecular weight greater than about 1 kDa, such as between about 1 kDa and about 1.000.000 kDa, such as between 25 kDa and 75 kDa.

20. The method according to claim 1, wherein said synthetic biodegradable scaffold is biocompatible.

21. The method according to claim 1, wherein said synthetic biodegradable scaffold is in the form selected from the group consisting of: a sheet, a membrane, a molded form, a plug, a tube, a sphere, a disc, granules, non-woven and woven fibres, freeze dried polymer such as freeze dried polymer sheets, or custom made three dimensional form of desired shape fitted for implantation into site of defect or site requiring implantation.

22. The method according to claim 1, wherein said synthetic biodegradable scaffold is part of a component which further comprises a biopolymer, such as a non-synthetic biopolymer, such as polysaccharides, polypeptides, lignin, polyphosphate or polyhydroxyalkanoates.

23. The method according to claim 22, wherein said biopolymer is selected from the group consisting of: gelatin, hyaluronan, hyaluronic acid (HA), dermatan sulphate, collagen, such as collagen type I and/or type II, alginate, chitin, chitosan, keratin, silk, cellulose and derivatives thereof, and agarose.

24. The method according to claim 22, wherein said biopolymer is any suitable extracellular matrix component.

25. The method according to claim 1 wherein said synthetic biodegradable scaffold comprises or consists of a compound selected from the group consisting of: a) Homo- or copolymers of: glycolide (polyglycolide, PGA), polylactide (PLA), such as L-lactide, DL-lactide, meso-lactide, ε-caprolactone (polycapro lactone, PCL), 1,4-dioxane-2-one, d-valerolactone, 1-butyrolactone, g-butyrolactone, e-decalactone, 1,4-dioxepane-2-one, 1,5-dioxepan-2-one, 1,5,8,12-tetraoxacyclotetradecane-7-14-dione, 1,5-dioxepane-2-one, 6,6-dimethyl-1,4-dioxane-2-one, and trimethylene carbonate; b) Block-copolymers of mono- or difunctional polyethylene glycol and polymers of a) mentioned above; c) Block copolymers of mono- or difunctional polyalkylene glycol and polymers of a) mentioned above; d) Blends of the above mentioned polymers; and e) polyanhydrides and polyorthoesters; such as copolymers of poly(D,L-lactide-co-glycolide) (PLGA), MPEG-PLGA (methoxypolyethyleneglycol)-poly(D,L-lactide-co-glycolide).

26. The method according to claim 25, wherein said synthetic biodegradable scaffold consists or comprises PLGA or MPEG-PLGA.

27. The method according to claim 26, wherein the MPEG-PLGA is a polymer of the general formula:
A-O— (CHR¹CHR²O)ₙ—B
wherein;
A is a poly(lactide-co-glycolide) residue of a molecular weight of at least 4000 g/mol, the molar ratio of (i) lactide units and (ii) glycolide units in the poly(lactide-co-glycolide) residue being in the range of 80:20 to 10:90;
B is either a poly(lactide-co-glycolide) residue as defined for A or is selected from the group consisting of hydrogen, C₁₋₆-alkyl and hydroxy protecting groups,
one of R¹ and R² within each —(CHR¹CHR²O)— unit is selected from hydrogen and methyl, and the other of R¹ and R² within the same —(CHR¹CHR²O)— unit is hydrogen;
n represents the average number of —(CHR¹CHR²O)— units within a polymer chain and is an integer in the range of 10-1000; and wherein
the molar ratio of (iii) polyalkylene glycol units —(CHR¹CHR²O)— to the combined amount of (i) lactide units and (ii) glycolide units in the poly(lactide-co-glycolide) residue(s) is at the most 20:80;
and wherein the molecular weight of the copolymer is at least 10,000 g/mol, preferably at least 15,000 g/mol.

28. The method according to claim 27, wherein both of R¹ and R² within each unit are hydrogen.

29. The method according to claim 27, wherein B is a poly(lactide-co-glycolide) residue as defined for A.

30. The method according to claim 27, wherein B is C₁₋₆-alkyl.

31. The method according to claim 27, wherein B is a hydroxy protecting group.

32. The method according to claim 27, wherein B is a hydroxy group.

33. The method according to claim 25, wherein said synthetic biodegradable scaffold is prepared by freeze drying a solution comprising the compound in solution.

34. The method according to claim 25, wherein said synthetic biodegradable scaffold has porosity in the range of 50 to 97%.

35. The method according to claim 1, wherein said chondrogenic cells are applied and/or grown in the presence of a biologically acceptable fixative precursor, such as fibrinogen.

36. The method according to claim 35, wherein the fibrinogen is recombinantly prepared.

37. The method according to claim 35, wherein the fibrinogen is isolated from a mammalian host cell such as a host cell obtained or derived from the same species as the individual mammal, or a transgenic host.

38. The method according to claim 35, wherein the concentration of fibrinogen used is 1-100 mg/ml.

39. The method according to claim 1, wherein the chondrogenic cells are applied and/or grown in the presence of a conversion agent suitable of converting the fixative precursor into a fixative material.

40. The method according to claim 39, wherein said conversion agent is a cross-linking agent.

41. The method according to claim 39, wherein said conversion agent is selected from the group consisting of: thrombin, a thrombin analogue, recombinant thrombin or a recombinant thrombin analogue.

42. The method according to claim 41, wherein the concentration of thrombin used is between 0.1 NIH unit and 150 NIH units, and/or a suitable level of thrombin for polymerizing 1-100 mg/ml fibrinogen.

43. A biosynthetic cartilaginous matrix prepared by a method according to claim 1.

44. An isolated, acellular biosynthetic cartilaginous matrix substantially devoid of synthetic biodegradable scaffold structure.

45. An isolated acellular biosynthetic cartilaginous matrix substantially devoid of synthetic biodegradable scaffold structure, having a morphological structure substantially comparable with the morphological structure of a synthetic biodegradable scaffold as defined in claim 18.

46. A method for the treatment or for alleviating the symptoms of a cartilage defects in a living individual mammal, such as a human being, said method comprising the step of:
a) applying a biosynthetic cartilaginous matrix according to claim 42 to the site of said defect.

47. The method according to claim 46, wherein cells derived from said living individual mammal are applied to the biosynthetic cartilaginous matrix prior to and/or concomitantly with and/or subsequent to the application of the biosynthetic cartilaginous matrix to the site of defect.

48. The method according claim 46, wherein a microfracture is purposely induced under clinical conditions at the site of implantation prior to application of the biosynthetic cartilaginous matrix.

49. The method of treatment according to claim 46, wherein the cartilage defect is due to trauma, osteonecrosis, or osteochondritis, and located in a joint, such as in the knee joint, or located in the ankle, shoulder, elbow, hip or spinal cord.

50. The method of treatment according to claim 46, wherein said biosynthetic cartilaginous matrix are immuno-compatible with said living individual mammal.

51. The method of treatment according to claim 46, wherein the treatment is performed as part of surgery, such as of endoscopic, atheroscopic, or minimal invasive surgery, and conventional or major open surgery.

52. The method of treatment according to claim 46, wherein the treatment is performed as part of reconstruction surgery or cosmetic surgery.

53. A biosynthetic cartilaginous matrix according to claim 43; for use as a medicament.

54. A biosynthetic cartilaginous matrix according to claim 43; for use in the treatment or for alleviating the symptoms of a cartilage defects in a living individual mammal, such as a human being.

55. The biosynthetic cartilaginous matrix according to claim 53, wherein the cartilage defect is due to trauma, osteonecrosis, or osteochondritis, and located in a joint, such as in the knee joint, or located in the ankle, shoulder, elbow, hip or spinal cord.

56. The biosynthetic cartilaginous matrix according to claim 53, wherein said biosynthetic cartilaginous matrix are immuno-compatible with said living individual mammal.

57. The biosynthetic cartilaginous matrix according to claim 53, wherein the medicament is for treatment as part of surgery, such as of endoscopic, atheroscopic, or minimal invasive surgery, and conventional or major open surgery.

58. The biosynthetic cartilaginous matrix according to claim 53, wherein the medicament is for treatment as part of reconstruction surgery or cosmetic surgery.

59. A kit of parts, for the treatment or for alleviating the symptoms of a cartilage defects in a living individual mammal, said kit comprising a biosynthetic cartilaginous matrix according to claim 43 and instructions for use of said biosynthetic cartilaginous matrix.

60. A kit of parts, for the treatment or for alleviating the symptoms of a cartilage defects in a living individual mammal, which comprises an integrated supply device, comprising the following functionally linked devices: (i) at least one container which contains said biosynthetic cartilaginous matrix prepared by a method according to claim 1, (ii) a delivery device, wherein said delivery device is suitable for direct application of said biosynthetic cartilaginous matrix to the site of defect in a living mammalian tissue and (iii) instructions for use of said biosynthetic cartilaginous matrix.

61. The kit of parts according to claim 60, wherein said delivery device is in the form of a medical device selected from the group consisting of: a syringe, a catheter, a needle, and a tube, a spraying device and a pressure gun.
